(19) United States
(12) Patent Application Publication
(10) Pub. No.: US 2013/0225625 A1
(43) Pub. Date: Aug. 29, 2013

(54) TAMPER-RESISTANT PHARMACEUTICAL DOSAGE FORM COMPRISING NONIONIC SURFACTANT

(71) Applicant: Grunenthal GmbH, (US)

(72) Inventors: Lutz BARNSCHEID, Mönchengladbach (DE); Sebastian SCHWIER, Aachen (DE); Jessica Redmer, Mönchengladbach (DE)

(73) Assignee: GRUNENTHAL GMBH, Aachen (DE)

(21) Appl. No.: 13/778,179

(22) Filed: Feb. 27, 2013

Related U.S. Application Data
(60) Provisional application No. 61/603,984, filed on Feb. 28, 2012.

(30) Foreign Application Priority Data
Feb. 28, 2012 (EP) 12 001 296.8

(51) Int. Cl. A61K 47/34

(52) U.S. Cl. CPC A61K 47/34
USPC 514/282; 514/646; 514/654

(57) ABSTRACT

The invention relates to a pharmaceutical dosage form having a breaking strength of at least 500 N and comprising a pharmacologically active compound, a polyalkylene oxide having an average molecular weight of at least 200,000 g/mol, and a nonionic surfactant; wherein the content of the polyalkylene oxide is within the range of from 20 to 75 wt.-%, based on the total weight of the pharmaceutical dosage form.

This application claims priority of U.S. Provisional Patent Application No. 61/603,984, filed on Feb. 28, 2012, and European Patent Application No. 12 001 296.8, filed on Feb. 28, 2012, the entire contents of which patent applications are incorporated herein by reference.

The invention relates to a pharmaceutical dosage form having a breaking strength of at least 500 N and comprising a pharmacologically active compound, a polyalkylene oxide having an average molecular weight of at least 200,000 g/mol, and a nonionic surfactant; wherein the content of the polyalkylene oxide is within the range of from 20 to 75 wt.-%, based on the total weight of the pharmaceutical dosage form.

Tamper-resistant pharmaceutical dosage forms containing pharmacologically active compounds have been known for many years. Pharmacologically active compound abuse with conventional dosage forms is typically achieved by (i) pulverization of the pharmaceutical dosage form and nasal administration of the powder; (ii) pulverization of the pharmaceutical dosage form, dissolution of the powder in a suitable liquid and intravenous administration of the solution; (iii) pulverization of the pharmaceutical dosage form and inhalation by smoking; (iv) liquid extraction of the drug from the pharmaceutical dosage form and intravenous administration of the solution; and the like. Accordingly, many of these methods of abuse require the mechanical destruction of the pharmaceutical dosage form in order to render it suitable for abuse.

In the past several different methods have been developed to avoid drug abuse.

Some of these concepts of rendering pharmaceutical dosage forms tamper resistant rely on the mechanical properties of the pharmaceutical dosage forms, particularly a substantially increased breaking strength (resistance to crushing). The major advantage of such pharmaceutical dosage forms is that comminuting, particularly pulverization, by conventional means, such as grinding in a mortar or fracturing by means of a hammer, is impossible or at least substantially impeded. Thus, by conventional means that are available to an abuser, such pharmaceutical dosage forms cannot be converted into a form suitable for abuse, e.g. a powder for nasal administration. In this regard it can be referred to e.g., WO 2005/016313, WO 2005/016314, WO 2005/063214, WO 2005/102286, WO 2006/002883, WO 2006/002884, WO 2006/002886, WO 2006/082097, WO 2006/082099, and WO 2008/107149.

US 2011/020451 discloses a thermoformed pharmaceutical dosage form having a breaking strength of at least 300 N, comprising an opioid (A), a free physiologically acceptable acid (B) in an amount of from 0.001 to 5.0 wt.-%, based on the total weight of the pharmaceutical dosage form, and a polyalkylene oxide (C) having a weight average molecular weight Mw of at least 200,000 g/mol.

US 2010/203129 relates to pharmaceutical compositions, which provide controlled release of a drug. The compositions are said to be suitable for continuous administration as they remain effective throughout the treatment regimen.

US 2011/159100 discloses controlled release formulations and methods for preparing controlled release formulations for delivery of active drug substances. The formulations may be employed to produce pharmaceutical compositions, such as controlled release dosage forms, adjusted to a specific administration scheme.

These known tamper resistant pharmaceutical dosage forms, however, are not satisfactory in every respect and there is a demand for tamper resistant pharmaceutical dosage forms containing pharmacologically active compounds that have advantages compared to the tamper resistant pharmaceutical dosage forms of the prior art.

This object has been achieved by the subject-matter described hereinbelow.

A first aspect of the invention relates to a pharmaceutical dosage form having a breaking strength of at least 500 N and comprising a pharmacologically active compound, preferably opioid, a polyalkylene oxide having an average molecular weight of at least 200,000 g/mol, and a nonionic surfactant; wherein the content of the polyalkylene oxide is within the range of from 20 to 75 wt.-%, based on the total weight of the pharmaceutical dosage form.

It has been surprisingly found that liquid extraction of the pharmacologically active compound and subsequent administration of the thus obtained liquid by the non-prescribed, parenteral route can be substantially impeded by the presence of a nonionic surfactant. Furthermore, it has been surprisingly found that the specific mechanical properties of pharmaceutical dosage forms exhibiting a substantially increased resistance to crushing (breaking strength) are not significantly deteriorated by the presence of substantial amounts of nonionic surfactant.

### BRIEF DESCRIPTION OF THE DRAWINGS

The invention will now be described in greater detail with reference to the drawings, wherein:

FIG. 1 shows the in vitro release profiles of the pharmaceutical dosage forms according to Examples I-1′_{round}, I-2′_{round} and C-1′_{round}; and

FIGS. 2-A, 2-B, 2-C, 2-D and 2-E, respectively, show the force-displacement diagrams of examples C-1′_{round}, I-1′_{round}, I-2′_{round}, C-3 C-3_{round} and I-3_{round}.

Preferably, the pharmacologically active compound and the nonionic surfactant are homogeneously distributed over the pharmaceutical dosage form or, when the pharmaceutical dosage form comprises a film coating, over the coated core of the pharmaceutical dosage form. Preferably, the pharmacologically active compound and the nonionic surfactant are intimately mixed with one another and homogeneously dispersed in the polyalkylene oxide, preferably in molecular disperse form or solid disperse form. In other words, the pharmacologically active compound and the nonionic surfactant preferably form a solid solution or solid dispersion in the polyalkylene oxide.

Preferably, the pharmacologically active compound is not locally separated from the nonionic surfactant. Preferably, the pharmaceutical dosage form contains neither any subunits comprising pharmacologically active compound but no nonionic surfactant, nor any subunits comprising nonionic surfactant but no pharmacologically active compound.

Preferably, the pharmacologically active compound and the nonionic surfactant are embedded in a prolonged release matrix comprising the polyalkylene oxide. Thus, the prolonged release matrix is preferably a hydrophilic matrix. Preferably, the release profile of the pharmacologically active compound is matrix-retarded. Preferably, the pharmacologically active compound is embedded in a matrix comprising the polyalkylene oxide, said matrix controlling the release of the pharmacologically active compound from the pharmaceutical dosage form.

Physiologically acceptable materials which are known to the person skilled in the art may be used as supplementary matrix materials. Polymers, particularly preferably cellulose ethers and/or cellulose esters are preferably used as hydrophilic matrix materials. Ethylcellulose, hydroxypropylmethylcellulose, hydroxypropylcellulose, hydroxymethylcellulose, hydroxyethylcellulose, and/or the derivatives thereof, such as the salts thereof are very particularly preferably used as matrix materials. Other preferred polymers include polyacrylates, i.e. homopolymers or copolymers of acrylic acid or its salts, such as Carbopol® of various types.

Preferably, the relative weight ratio of the polyalkylene oxide to the pharmacologically active compound is at least 0.5:1, more preferably at least 1:1, at least 2:1, at least 3:1, at least 4:1, at least 5:1, at least 6:1, at least 7:1, at least 8:1 or at least 9:1. In a preferred embodiment, the relative weight ratio of the polyalkylene oxide to the pharmacologically active compound is within the range of from 5:1 to 1:1, more preferably 4:1 to 2:1. In another preferred embodiment, the relative weight ratio of the polyalkylene oxide to the pharmacologically active compound is within the range of from 2:1 to 1:1.

In a preferred embodiment, the pharmaceutical dosage form according to the invention is adapted for administration once daily, preferably orally. In another preferred embodiment, the pharmaceutical dosage form according to the invention is adapted for administration twice daily, preferably orally. In still another preferred embodiment, the pharmaceutical dosage form according to the invention is adapted for administration thrice daily, preferably orally.

For the purpose of the specification, “twice daily” means equal or nearly equal time intervals, i.e., about every 12 hours, or different time intervals, e.g., 8 and 16 hours or 10 and 14 hours, between the individual administrations.

For the purpose of the specification, “thrice daily” means equal or nearly equal time intervals, i.e., about every 8 hours, or different time intervals, e.g., 6, 6 and 12 hours; or 7, 7 and 10 hours, between the individual administrations.

Preferably, the pharmaceutical dosage form according to the invention causes an at least partially delayed or prolonged release of pharmacologically active compound.

Controlled or prolonged release is understood according to the invention preferably to mean a release profile in which the pharmacologically active compound is released over a relatively long period with reduced intake frequency with the purpose of extended therapeutic action of the pharmacologically active compound. Preferably, the meaning of the term “prolonged release” is in accordance with the European guideline on the nomenclature of the release profile of pharmaceutical dosage forms (CHMP). This is achieved in particular with peroral administration. The expression “at least partially delayed or prolonged release” covers according to the invention any pharmaceutical dosage forms which ensure modified release of the pharmacologically active compound contained therein. The pharmaceutical dosage forms preferably comprise coated or uncoated pharmaceutical dosage forms, which are produced with specific auxiliary substances, by particular processes or by a combination of the two possible options in order purposefully to change the release rate or location of release.

In the case of the pharmaceutical dosage forms according to the invention, the release profile of a controlled release form may be modified e.g. as follows: extended release, repeat action release, prolonged release and sustained release.

For the purpose of the specification “controlled release” preferably means a product in which the release of active compound over time is controlled by the type and composition of the formulation. For the purpose of the specification “extended release” preferably means a product in which the release of active compound is delayed for a finite lag time, after which release is unhindered. For the purpose of the specification “repeat action release” preferably means a product in which a first portion of active compound is released initially, followed by at least one further portion of active compound being released subsequently. For the purpose of the specification “prolonged release” preferably means a product in which the rate of release of active compound from the formulation after administration has been reduced over time, in order to maintain therapeutic activity, to reduce toxic effects, or for some other therapeutic purpose. For the purpose of the specification “sustained release” preferably means a way of formulating a medicine so that it is released into the body steadily, over a long period of time, thus reducing the dosing frequency. For further details, reference may be made, for example, to K. H. Bauer, Lehrbuch der Pharmazeutischen Technologie, 6th edition, WVG Stuttgart, 1999; and Eur. Ph.

The pharmaceutical dosage form according to the invention may comprise one or more pharmacologically active compounds at least in part in a further controlled release form, wherein controlled release may be achieved with the assistance of conventional materials and processes known to the person skilled in the art, for example by embedding the substances in a controlled release matrix or by applying one or more controlled release coatings. Substance release must, however, be controlled such that addition of delayed-release materials does not impair the necessary breaking strength. Controlled release from the pharmaceutical dosage form according to the invention is preferably achieved by embedding the pharmacologically active compound in a matrix. Preferably, the polyalkylene oxide serves as matrix material. The auxiliary substances acting as matrix materials control release. Matrix materials may, for example, be hydrophilic, gel-forming materials, from which release proceeds mainly by erosion and diffusion.

Preferably, the release profile is substantially matrix controlled, preferably by embedding the pharmacologically active compound in a matrix comprising the polyalkylene oxide and optionally, further matrix materials. Preferably, the release profile is not osmotically driven. Preferably, release kinetics is not zero order.

In preferred embodiments, in accordance with Ph. Eur., the in vitro release profile of the pharmacologically active compound complies with any same single one of the following release profiles R¹ to R⁶⁰:

| % | R¹ | R² | R³ | R⁴ | R⁵ | R⁶ | R⁷ | R⁸ | R⁹ | R¹⁰ |
|---|---|---|---|---|---|---|---|---|---|---|
| 1 h | 30 ± 28 | 30 ± 26 | 30 ± 24 | 30 ± 22 | 30 ± 20 | 30 ± 18 | 30 ± 16 | 30 ± 14 | 30 ± 12 | 30 ± 10 |
| 2 h | 45 ± 40 | 45 ± 38 | 45 ± 36 | 45 ± 34 | 45 ± 32 | 45 ± 30 | 45 ± 28 | 45 ± 26 | 45 ± 24 | 45 ± 24 |
| 4 h | 60 ± 35 | 60 ± 33 | 60 ± 31 | 60 ± 29 | 60 ± 27 | 60 ± 25 | 60 ± 23 | 60 ± 21 | 60 ± 19 | 60 ± 17 |
| 6 h | 70 ± 30 | 70 ± 28 | 70 ± 25 | 70 ± 23 | 70 ± 21 | 70 ± 19 | 70 ± 17 | 70 ± 15 | 70 ± 13 | 70 ± 11 |
| 8 h | ≧60 | 85 ± 13 | 85 ± 12 | 85 ± 11 | 85 ± 10 | 85 ± 9 | 85 ± 8 | 85 ± 7 | 85 ± 6 | 85 ± 5 |
| 10 h | ≧70 | ≧72 | ≧74 | ≧76 | ≧78 | ≧80 | ≧82 | ≧84 | ≧86 | ≧88 |
| 12 h | ≧80 | ≧82 | ≧84 | ≧86 | ≧88 | ≧90 | ≧92 | ≧94 | ≧96 | ≧98 |
| % | R¹¹ | R¹² | R¹³ | R¹⁴ | R¹⁵ | R¹⁶ | R¹⁷ | R¹⁸ | R¹⁹ | R²⁰ |
| 1 h | 40 ± 38 | 40 ± 36 | 40 ± 34 | 40 ± 32 | 40 ± 30 | 40 ± 28 | 40 ± 26 | 40 ± 24 | 40 ± 22 | 40 ± 20 |
| 2 h | 55 ± 43 | 55 ± 41 | 55 ± 39 | 55 ± 37 | 55 ± 35 | 55 ± 33 | 55 ± 31 | 55 ± 29 | 55 ± 27 | 55 ± 25 |
| 4 h | 70 ± 28 | 70 ± 26 | 70 ± 24 | 70 ± 22 | 70 ± 20 | 70 ± 18 | 70 ± 16 | 70 ± 14 | 70 ± 12 | 70 ± 10 |
| 6 h | 80 ± 20 | 80 ± 18 | 80 ± 16 | 80 ± 15 | 80 ± 14 | 80 ± 13 | 80 ± 12 | 80 ± 11 | 80 ± 10 | 80 ± 9 |
| 8 h | ≧80 | 90 ± 8 | 90 ± 8 | 90 ± 7 | 90 ± 7 | 90 ± 6 | 90 ± 6 | 90 ± 5 | 90 ± 5 | 90 ± 4 |
| 10 h | ≧85 | ≧87 | ≧89 | ≧90 | ≧90 | ≧91 | ≧91 | ≧92 | ≧92 | ≧92 |
| 12 h | ≧90 | ≧91 | ≧91 | ≧91 | ≧92 | ≧92 | ≧92 | ≧93 | ≧93 | ≧93 |
| % | R²¹ | R²² | R²³ | R²⁴ | R²⁵ | R²⁶ | R²⁷ | R²⁸ | R²⁹ | R³⁰ |
| 1 h | 20 ± 18 | 20 ± 16 | 20 ± 14 | 20 ± 13 | 20 ± 12 | 20 ± 11 | 20 ± 10 | 20 ± 9 | 20 ± 8 | 20 ± 7 |
| 2 h | 35 ± 33 | 35 ± 31 | 35 ± 30 | 35 ± 29 | 35 ± 27 | 35 ± 25 | 35 ± 23 | 35 ± 21 | 35 ± 19 | 35 ± 17 |
| 4 h | 50 ± 48 | 50 ± 46 | 50 ± 44 | 50 ± 42 | 50 ± 40 | 50 ± 38 | 50 ± 36 | 50 ± 34 | 50 ± 32 | 50 ± 31 |
| 6 h | 60 ± 38 | 60 ± 36 | 60 ± 34 | 60 ± 32 | 60 ± 30 | 60 ± 28 | 60 ± 26 | 60 ± 24 | 60 ± 22 | 60 ± 20 |
| 8 h | ≧60 | 70 ± 28 | 70 ± 26 | 70 ± 24 | 70 ± 22 | 70 ± 20 | 70 ± 18 | 70 ± 16 | 70 ± 14 | 70 ± 12 |
| 10 h | ≧70 | ≧72 | ≧74 | ≧76 | ≧78 | ≧80 | ≧82 | ≧84 | ≧86 | ≧88 |
| 12 h | ≧80 | ≧82 | ≧84 | ≧86 | ≧88 | ≧90 | ≧91 | ≧92 | ≧93 | ≧93 |
| % | R³¹ | R³² | R³³ | R³⁴ | R³⁵ | R³⁶ | R³⁷ | R³⁸ | R³⁹ | R⁴⁰ |
| 1 h | 8 ± 7 | 8 ± 6 | 8 ± 5 | 8 ± 4 | 13 ± 12 | 13 ± 10 | 13 ± 8 | 13 ± 6 | 18 ± 17 | 18 ± 14 |
| 2 h | 15 ± 14 | 15 ± 11 | 15 ± 8 | 15 ± 5 | 24 ± 23 | 24 ± 18 | 24 ± 13 | 24 ± 8 | 33 ± 32 | 33 ± 24 |
| 4 h | 30 ± 29 | 30 ± 22 | 30 ± 15 | 30 ± 8 | 38 ± 37 | 38 ± 28 | 38 ± 18 | 38 ± 8 | 55 ± 34 | 55 ± 26 |
| 6 h | 50 ± 49 | 50 ± 37 | 50 ± 25 | 50 ± 13 | 60 ± 39 | 60 ± 29 | 60 ± 19 | 60 ± 9 | 70 ± 29 | 70 ± 22 |
| 8 h | 65 ± 34 | 65 ± 26 | 65 ± 18 | 65 ± 10 | 75 ± 24 | 75 ± 18 | 75 ± 12 | 75 ± 6 | 83 ± 16 | 83 ± 13 |
| 10 h | 85 ± 14 | 85 ± 11 | 85 ± 8 | 85 ± 5 | 87 ± 12 | 87 ± 10 | 87 ± 8 | 87 ± 6 | 90 ± 9 | 90 ± 8 |
| 12 h | >95 | >95 | >95 | >95 | >95 | >95 | >95 | >95 | >95 | >95 |
| % | R⁴¹ | R⁴² | R⁴³ | R⁴⁴ | R⁴⁵ | R⁴⁶ | R⁴⁷ | R⁴⁸ | R⁴⁹ | R⁵⁰ |
| 1 h | 18 ± 11 | 18 ± 8 | 25 ± 24 | 25 ± 18 | 25 ± 12 | 25 ± 6 | 40 ± 39 | 40 ± 29 | 40 ± 19 | 40 ± 9 |
| 2 h | 33 ± 16 | 33 ± 8 | 45 ± 44 | 45 ± 33 | 45 ± 22 | 45 ± 11 | 63 ± 26 | 63 ± 20 | 63 ± 14 | 63 ± 8 |
| 4 h | 55 ± 18 | 55 ± 10 | 70 ± 29 | 70 ± 22 | 70 ± 15 | 70 ± 8 | 85 ± 14 | 85 ± 12 | 85 ± 10 | 85 ± 8 |
| 6 h | 70 ± 15 | 70 ± 8 | 83 ± 16 | 83 ± 13 | 83 ± 10 | 83 ± 7 | 90 ± 9 | 90 ± 8 | 90 ± 7 | 90 ± 6 |
| 8 h | 83 ± 10 | 83 ± 7 | 92 ± 7 | 92 ± 6 | 92 ± 6 | 92 ± 5 | 92 ± 7 | 92 ± 7 | 92 ± 6 | 92 ± 6 |
| 10 h | 90 ± 7 | 90 ± 6 | 94 ± 6 | 94 ± 6 | 94 ± 5 | 94 ± 5 | 94 ± 6 | 94 ± 6 | 94 ± 5 | 94 ± 5 |
| 12 h | >95 | >95 | >95 | >95 | >95 | >95 | >95 | >95 | >95 | >95 |
| % | R⁵¹ | R⁵² | R⁵³ | R⁵⁴ | R⁵⁵ | R⁵⁶ | R⁵⁷ | R⁵⁸ | R⁵⁹ | R⁶⁰ |
| 1 h | 18 ± 11 | 18 ± 8 | 18 ± 62 | 5 ± 18 | 25 ± 12 | 25 ± 9 | 40 ± 39 | 40 ± 29 | 40 ± 19 | 40 ± 9 |
| 2 h | 25 ± 16 | 25 ± 10 | 25 ± 8 | 35 ± 22 | 35 ± 18 | 35 ± 15 | 50 ± 26 | 50 ± 24 | 50 ± 18 | 50 ± 12 |
| 8 h | 55 ± 18 | 55 ± 12 | 55 ± 8 | 65 ± 25 | 65 ± 18 | 65 ± 12 | 75 ± 23 | 75 ± 20 | 75 ± 14 | 75 ± 10 |
| 12 h | 70 ± 15 | 70 ± 10 | 70 ± 6 | 80 ± 18 | 80 ± 15 | 80 ± 10 | 90 ± 8 | 90 ± 6 | 90 ± 5 | 90 ± 5 |
| 24 h | >85 | ≧88 | >90 | >90 | >90 | ≧95 | >90 | >90 | >95 | >95 |

Suitable in vitro conditions are known to the skilled artisan. In this regard it can be referred to, e.g., the Ph. Eur. Preferably, the in vitro release profile is measured under the following conditions: 600 ml phosphate buffer (pH 6.8) at temperature of 37° C. with sinker (type 1 or 2); rotation speed of the paddle: 75 min⁻¹.

Preferably, the release profile of the pharmaceutical dosage form according to the invention is stable upon storage, preferably upon storage at elevated temperature, e.g. 37° C., for 3 months in sealed containers. In this regard “stable” means that when comparing the initial release profile with the release profile after storage, at any given time point the release profiles deviate from one another absolutely by not more than 20%, more preferably not more than 15%, still more preferably not more than 10%, yet more preferably not more than 7.5%, most preferably not more than 5.0% and in particular not more than 2.5%.

Preferably, the pharmaceutical dosage form according to the invention is monolithic. Preferably, the pharmaceutical dosage form is a monolithic mass. The pharmaceutical dosage form is preferably prepared by hot-melt extrusion. The melt extruded strands are preferably cut into monoliths, which are then preferably formed into tablets. In this regard, the term “tablets” is preferably not to be understood as pharmaceutical dosage forms being made by compression of powder or granules (compressi) but rather, as shaped extrudates.

The pharmaceutical dosage form according to the invention comprises a polyalkylene oxide having a weight average molecular weight M_{w} of at least 200,000 g/mol, preferably at least 500,000 g/mol, more preferably at least 750,000 g/mol, still more preferably at least 1,000,000 g/mol, yet more preferably at least 1,500,000 g/mol, most preferably at least 2,000,000 g/mol and in particular within the range of from 500,000 to 15,000,000 g/mol.

Preferably, the polyalkylene oxide is selected from the group consisting of polymethylene oxide, polyethylene oxide and polypropylene oxide, the copolymers and mixtures thereof.

Polyalkylene oxide may comprise a single polyalkylene oxide having a particular average molecular weight, or a mixture (blend) of different polymers, such as two, three, four or five polymers, e.g., polymers of the same chemical nature but different average molecular weight, polymers of different chemical nature but same average molecular weight, or polymers of different chemical nature as well as different molecular weight.

For the purpose of the specification, a polyalkylene glycol has a molecular weight of up to 20,000 g/mol whereas a polyalkylene oxide has a molecular weight of more than 20,000 g/mol. In a preferred embodiment, the weight average over all molecular weights of all polyalkylene oxides that are contained in the pharmaceutical dosage form is at least 200,000 g/mol. Thus, polyalkylene glycols, if any, are preferably not taken into consideration when determining the weight average molecular weight of polyalkylene oxide.

The content of the polyalkylene oxide is within the range of from 20 to 75 wt.-%, based on the total weight of the pharmaceutical dosage form.

In another preferred embodiment, the content of the polyalkylene oxide is within the range of from 30 to 75 wt.-%, based on the total weight of the pharmaceutical dosage form. In a preferred embodiment, the content of the polyalkylene oxide is at least 25 wt.-%, still more preferably at least 30 wt.-%, yet more preferably at least 35 wt.-% and in particular at least 40 wt.-%, based on the total weight of the pharmaceutical dosage form.

In a preferred embodiment, the overall content of polyalkylene oxide is within the range of 25±5 wt.-%. In another preferred embodiment, the overall content of polyalkylene oxide is within the range of 35±15 wt.-%, most preferably 35±10 wt.-%, and in particular 35±5 wt.-%. In still another preferred embodiment, the overall content of polyalkylene oxide is within the range of 45±20 wt.-%, more preferably 45±15 wt.-%, most preferably 45±10 wt.-%, and in particular 45±5 wt.-%. In yet another preferred embodiment, the overall content of polyalkylene oxide is within the range of 55±20 wt.-%, more preferably 55±15 wt.-%, most preferably 55±10 wt.-%, and in particular 55±5 wt.-%. In a further preferred embodiment, the overall content of polyalkylene oxide is within the range of 65±10 wt.-%, and in particular 65±5 wt.-%.

In a preferred embodiment, the polyalkylene oxide is homogeneously distributed in the pharmaceutical dosage form according to the invention. Preferably, the polyalkylene oxide forms a matrix in which the pharmacologically active compound and the nonionic surfactant are embedded. In a particularly preferred embodiment, the pharmacologically active compound, the nonionic surfactant and the polyalkylene oxide are intimately homogeneously distributed in the pharmaceutical dosage form so that the pharmaceutical dosage form does not contain any segments where either pharmacologically active compound is present in the absence of nonionic surfactant and/or polyalkylene oxide, or where nonionic surfactant is present in the absence of pharmacologically active compound and/or polyalkylene oxide or where polyalkylene oxide is present in the absence of pharmacologically active compound and/or nonionic surfactant.

When the pharmaceutical dosage form is film coated, the polyalkylene oxide is preferably homogeneously distributed in the core of the pharmaceutical dosage form, i.e. the film coating preferably does not contain polyalkylene oxide. Nonetheless, the film coating as such may of course contain one or more polymers, which however, preferably differ from the polyalkylene oxide contained in the core.

The polyalkylene oxide may be combined with one or more different polymers selected from the group consisting of polyalkylene oxide, preferably polymethylene oxide, polyethylene oxide, polypropylene oxide; polyethylene, polypropylene, polyvinyl chloride, polycarbonate, polystyrene, polyvinylpyrrolidone, poly(hydroxy fatty acids), such as for example poly(3-hydroxybutyrate-co-3-hydroxyvalerate) (Biopol®), poly(hydroxyvaleric acid); polycaprolactone, polyvinyl alcohol, polyesteramide, polyethylene succinate, polylactone, polyglycolide, polyurethane, polyamide, polylactide, polyacetal (for example polysaccharides optionally with modified side chains), polylactide/glycolide, polylactone, polyglycolide, polyorthoester, polyanhydride, block polymers of polyethylene glycol and polybutylene terephthalate (Polyactive®), polyanhydride (Polifeprosan), copolymers thereof, block-copolymers thereof, and mixtures of at least two of the stated polymers, or other polymers with the above characteristics. Other preferred polymers include polyacrylates, i.e. homopolymers or copolymers of acrylic acid or its salts, such as Carbopol® of various types.

Preferably, the molecular weight dispersity M_{w}/Mₙ of polyalkylene oxide is within the range of 2.5±2.0, more preferably 2.5±1.5, still more preferably 2.5±1.0, yet more preferably 2.5±0.8, most preferably 2.5±0.6, and in particular 2.5±0.4.

The polyalkylene oxide preferably has a viscosity at 25° C. of 30 to 17,600 cP, more preferably 55 to 17,600 cP, still more preferably 600 to 17,600 cP and most preferably 4,500 to 17,600 cP, measured in a 5 wt.-% aqueous solution using a model RVF Brookfield viscosimeter (spindle no. 2/rotational speed 2 rpm); of 400 to 4,000 cP, more preferably 400 to 800 cP or 2,000 to 4,000 cP, measured on a 2 wt.-% aqueous solution using the stated viscosimeter (spindle no. 1 or 3/rotational speed 10 rpm); or of 1,650 to 10,000 cP, more preferably 1,650 to 5,500 cP, 5,500 to 7,500 cP or 7,500 to 10,000 cP, measured on a 1 wt.-% aqueous solution using the stated viscosimeter (spindle no. 2/rotational speed 2 rpm).

In a preferred embodiment, the prolonged release matrix comprises an additional matrix polymer.

In a preferred embodiment according to the invention, the polyalkylene oxide having a weight average molecular weight of at least 200,000 g/mol is combined with at least one further polymer, preferably but not necessarily also having a weight average molecular weight (M_{w}) of at least 200,000 g/mol, selected from the group consisting of polyethylene, polypropylene, polyvinyl chloride, polycarbonate, polystyrene, poly(hydroxy fatty acids), polycaprolactone, polyvinyl alcohol, polyesteramide, polyethylene succinate, polylactone, polyglycolide, polyurethane, polyvinylpyrrolidone, polyamide, polylactide, polylactide/glycolide, polylactone, polyglycolide, polyorthoester, polyanhydride, block polymers of polyethylene glycol and polybutylene terephthalate, polyanhydride, polyacetal, cellulose esters, cellulose ethers and copolymers thereof. Cellulose esters and cellulose ethers are particularly preferred, e.g. methylcellulose, ethylcellulose, hydroxymethylcellulose, hydroxyethylcellulose, hydroxypropylcellulose hydroxypropylmethylcellulose, carboxymethylcellulose, and the like. Other preferred polymers include polyacrylates, i.e. homopolymers or copolymers of acrylic acid or its salts, such as Carbopol® of various types.

In a preferred embodiment, said further polymer is neither a polyalkylene oxide nor a polyalkylene glycol. Nonetheless, the pharmaceutical dosage form may contain polyalkylene glycol, e.g. as plasticizer, but then, the pharmaceutical dosage form preferably is an at least ternary mixture of polymers: polyalkylene oxide+further polymer+plasticizer.

In a particularly preferred embodiment, said further polymer is a hydrophilic cellulose ester or cellulose ether, preferably hydroxypropylmethylcellulose (HPMC), hydroxypropylcellulose (HPC) or hydroxyethylcellulose (HEC), preferably having an average viscosity (preferably measured by capillary viscosimetry or rotational viscosimetry) of 1,000 to 150,000 mPas, more preferably 3,000 to 150,000. In a preferred embodiment, the average viscosity is within the range of 110,000±50,000 mPas, more preferably 110,000±40,000 mPas, still more preferably 110,000±30,000 mPas, most preferably 110,000±20,000 mPas, and in particular 100,000±10,000 mPas.

In a preferred embodiment, the further polymer is a cellulose ester or cellulose ether, preferably HPMC, having a content within the range of 10±8 wt.-%, more preferably 10±6 wt.-%, still more preferably 10±5 wt.-%, yet more preferably 10±4 wt.-%, most preferably 10±3 wt.-%, and in particular 10±2 wt.-%, based on the total weight of the pharmaceutical dosage form.

In another preferred embodiment, the further polymer is a cellulose ester or cellulose ether, preferably HPMC, having a content within the range of 15±8 wt.-%, more preferably 15±6 wt.-%, still more preferably 15±5 wt.-%, yet more preferably 15±4 wt.-%, most preferably 15±3 wt.-%, and in particular 15±2 wt.-%, based on the total weight of the pharmaceutical dosage form.

In another, particularly preferred embodiment, the pharmaceutical dosage form according to the invention, in addition to the polyalkylene oxide, contains a further polymer obtainable by polymerization of a monomer composition comprising an ethylenically unsaturated monomer bearing an anionic functional group, in protonated form or a physiologically acceptable salt thereof. The pharmacologically active compound is then preferably embedded into a controlled-release matrix comprising the polyalkylene oxide as well as said further polymer.

Preferably, the anionic functional group is selected from carboxyl groups, sulfonyl groups, sulfate groups, and phosphoryl groups.

Preferably, the monomer composition comprises an ethylenically unsaturated monomer selected from ethylenically unsaturated carboxylic acids, ethylenically unsaturated carboxylic acid anhydrides, ethylenically unsaturated sulfonic acids and mixtures thereof.

Preferred ethylenically unsaturated carboxylic acid and ethylenically unsaturated carboxylic acid anhydride monomers include the acrylic acids typified by acrylic acid itself, methacrylic acid, ethacrylic acid, alpha-chloracrylic acid, alpha-cyano acrylic acid, beta-methyl-acrylic acid (crotonic acid), alpha-phenyl acrylic acid, beta-acryloxy propionic acid, sorbic acid, alpha-chloro sorbic acid, angelic acid, cinnamic acid, p-chloro cinnamic acid, beta-styryl acrylic acid (1-carboxy-4-phenyl butadiene-1,3), itaconic acid, citraconic acid, mesaconic acid, glutaconic acid, aconitic acid, maleic acid, fumaric acid, tricarboxy ethylene and maleic acid anhydride.

Preferred ethylenically unsaturated sulfonic acids include aliphatic or aromatic vinyl sulfonic acids such as vinylsulfonic acid, allyl sulfonic acid, vinyltoluenesulfonic acid and styrene sulfonic acid; acrylic and methacrylic sulfonic acid such as sulfoethyl acrylate, sulfoethyl methacrylate, sulfopropyl acrylate, sulfopropyl methacrylate, 2-hydroxy-3-acryloxy propyl sulfonic acid, 2-hydroxy-3-methacryloxy propyl sulfonic acid and 2-acrylamido-2-methyl propane sulfonic acid.

Preferably, the monomer composition comprises acrylic acid, methacrylic acid, and/or 2-acrylamido-2-methyl propane sulfonic acid. Acrylic acid is especially preferred.

The further polymer is obtainable by polymerization of such a monomer composition. This does not necessarily require that it has been obtained from such a monomer composition indeed. In other words, the further polymer is a polymer comprising at least one repeating unit which results from polymerization of an ethylenically unsaturated monomer bearing an anionic functional group, in protonated form or a physiologically acceptable salt thereof.

The further polymer may be linear or branched or cross-linked.

Preferably, further polymer is hydrophilic, more preferably water-soluble or water-swellable.

The further polymer may be a homopolymer or a copolymer. When further polymer is a homopolymer, it comprises a single type of repeating unit, i.e. is the polymerization product of a monomer composition comprising a single type of monomer. When further polymer is a copolymer, it may comprise two, three or more different repeating units, i.e. may be the polymerization product of a monomer composition comprising two, three or more different monomers.

In a preferred embodiment, the further polymer is a copolymer, comprising from about 50 mol-% to 99.999 mol-%, and more preferably from about 75 mol-% to 99.99 mol-% repeating units bearing anionic functional groups, preferably acid groups, more preferably carboxylic groups.

Preferably, the further polymer has an average equivalent weight of 76±50 g/mol, more preferably of 76±30 g/mol, still more preferably of 76±20 g/mol and most preferably of 76±10 g/mol per carboxyl group.

In a preferred embodiment, the monomer composition from which further polymer is derivable, further comprises a cross-linking agent, i.e. in this embodiment the further polymer is cross-linked.

Suitable cross-linking agents include
compounds having at least two polymerizable double bonds, e.g. ethylenically unsaturated functional groups;
compounds having at least one polymerizable double bond, e.g. an ethylenically unsaturated functional group, and at least one functional group that is capable of reacting with another functional group of one or more of the repeating units of further polymer;
compounds having at least two functional groups that are capable of reacting with other functional groups of one or more of the repeating units of further polymer; and
polyvalent metal compounds which can form ionic cross-linkages, e.g. through the anionic functional groups.

Cross-linking agents having at least two polymerizable double bonds, preferably allyl groups, are particularly preferred.

Cross-linking agents having at least two polymerizable double bonds include (i) di- or polyvinyl compounds such as divinylbenzene and divinyltoluene; (ii) di- or poly-esters of unsaturated mono- or poly-carboxylic acids with polyols including, for example, di- or triacrylic acid esters of polyols such as ethylene glycol, trimethylol propane, glycerine, or polyoxyethylene glycols; (iii) bisacrylamides such as N,N-methylenebisacrylamide; (iv) carbamyl esters that can be obtained by reacting polyisocyanates with hydroxyl group-containing monomers; (v) di- or poly-allyl ethers of polyols; (vi) di- or poly-allyl esters of polycarboxylic acids such as diallyl phthalate, diallyl adipate, and the like; (vii) esters of unsaturated mono- or poly-carboxylic acids with mono-allyl esters of polyols such as acrylic acid ester of polyethylene glycol monoallyl ether; and (viii) di- or triallyl amine.

In a preferred embodiment, divinyl glycol (1,5-hexadiene-3,4-diol) is contained as cross-linking agent, whereas allyl or vinyl derivatives of polyols, such as allylsucrose or allyl pentaerythritol, are less preferred. This embodiment is preferably realized by polyacrylic acid polymers of polycarbophil type according to USP.

In another preferred embodiment, allyl derivatives of polyols, such as allylsucrose or allyl pentaerythritol, are contained as cross-linking agent, whereas divinyl glycol (1,5-hexadiene-3,4-diol) is less preferred. This embodiment is preferably realized by polyacrylic acid polymers of carbomer type according to USP or Ph. Eur.

Cross-linking agents having at least one polymerizable double bond and at least one functional group capable of reacting with other functional groups of one or more of the repeating units of further polymer include N-methylol acrylamide, glycidyl acrylate, and the like.

Suitable cross-linking agents having at least two functional groups capable of reacting with other functional groups of one or more of the repeating units of further polymer include glyoxal; polyols such as ethylene glycol; polyamines such as alkylene diamines (e.g., ethylene diamine), polyalkylene polyamines, polyepoxides, di- or polyglycidyl ethers and the like.

Suitable polyvalent metal cross-linking agents which can form ionic cross-linkages include oxides, hydroxides and weak acid salts (e.g., carbonate, acetate and the like) of alkaline earth metals (e.g., calcium magnesium) and zinc, including, for example, calcium oxide and zinc diacetate.

Of all of these types of cross-linking agents, the most preferred for use herein are diol derivatives and polyol derivatives, more specifically those selected from the group consisting of allyl sucrose, allyl pentaerythritol, divinyl glycol, divinyl polyethylene glycol and (meth)acrylic acid esters of diols.

In a preferred embodiment, the monomer composition from which the further polymer is derivable comprises the cross-linking agent in an amount of at most 1.0 mol-%, more preferably at most 0.1 mol-%, even more preferably at most about 0.01 mol-%, and most preferably at most 0.005 mol-% based on all monomers forming further polymer.

In a preferred embodiment, further polymer is a homopolymer of acrylic acid, optionally cross-linked, preferably with allyl sucrose or allyl pentaerythritol, in particular with allyl pentaerythritol. In another preferred embodiment, further polymer is a copolymer of acrylic acid and C₁₀-C₃₀-alkyl acrylate, optionally cross-linked, preferably with allyl pentaerythritol. In another preferred embodiment, further polymer is a so-called interpolymer, namely a homopolymer of acrylic acid, optionally cross-linked, preferably with allyl sucrose or allyl pentaerythritol; or a copolymer of acrylic acid and C₁₀-C₃₀-alkyl acrylate, optionally cross-linked, preferably with allyl pentaerythritol; which contain a block copolymer of polyethylene glycol and a long chain alkyl acid, preferably a C₈-C₃₀-alkyl acid. Polymers of this type are commercially available, e.g. under the trademark Carbopol®.

In another preferred embodiment, further polymer, preferably the pharmaceutical dosage form according to the invention does not contain a block copolymer of polyethylene glycol and an alkyl acid ester.

When further polymer is an interpolymer, it preferably has a viscosity in 1.0 wt.-% solution at pH 7.5 within the range of from 47,000 to 77,000 mPa·s, more preferably 52,000 to 72,000 mPa·s, still more preferably 57,000 to 67,000 mPa·s.

Preferably, at least some of the anionic functional groups contained in the further polymer are present in neutralized form, i.e. they are not present in their protonated forms, but are salts with salt-forming cations instead. Suitable salt-forming cations include alkali metal, ammonium, substituted ammonium and amines. More preferably, at least some of the anionic functional groups, e.g. carboxylate and/or sulfonate anions, are salts of sodium or potassium cations.

This percentage of neutralized anionic functional groups, based on the total amount of anionic functional groups, is referred to herein as the “degree of neutralization.” In a preferred embodiment, the degree of neutralization is within the range of from 2.5±2.4%, more preferably 2.5±2.0%, still more preferably 2.5±1.5%, yet more preferably 2.5±1.0%, and most preferably 2.5±0.5%. In another preferred embodiment, the degree of neutralization is within the range of 35±30%, more preferably 35±25%, still more preferably 35±20%, yet more preferably 35±15%, most preferably 35±10%, and in particular 35±5%. In yet another preferred embodiment, the degree of neutralization is in the range of 65±30%, more preferably 65±25%, still more preferably 65±20%, yet more preferably 65±15%, most preferably 65±10%, and in particular 65±5%.

The content of further polymer ranges preferably from 0.1 wt.-% to 95 wt.-%, more preferably from 1.0 wt.-% to 80 wt.-%, still more preferably from 2.0 wt.-% to 50 wt.-%, and most preferably from 5 wt.-% to 30% wt.-%, and in particular 9 wt.-% to 25 wt.-%, based on the total weight of the pharmaceutical dosage form.

In a preferred embodiment, the content of further polymer amounts to 0.5 to 25 wt.-%, more preferably 1.0 to 20 wt.-%, still more preferably 2.0 to 22.5 wt.-%, yet more preferably 3.0 to 20 wt.-% and most preferably 4.0 to 17.5 wt.-% and in particular 5.0 to 15 wt.-%, based on the total weight of the pharmaceutical dosage form.

In another preferred embodiment, the content of further polymer amounts to 0.5 to 40 wt.-%, more preferably 5 to 35 wt.-%, still more preferably 7.5 to 30 wt.-%, yet more preferably 10 to 30 wt.-% and most preferably 15 to 25 wt.-% and in particular 17.5 to 25 wt.-%, based on the total weight of the pharmaceutical dosage form.

In a preferred embodiment, the content of further polymer is within the range of 10±9 wt.-%, more preferably 10±8 wt.-%, still more preferably 10±7 wt.-%, yet more preferably 10±6 wt.-%, most preferably 10±5 wt.-%, and in particular 10±2.5 wt.-%, based on the total weight of the pharmaceutical dosage form.

In still another preferred embodiment, the content of further polymer is within the range of 15±14 wt.-%, more preferably 15±12.5 wt.-%, still more preferably 15±10 wt.-%, yet more preferably 15±7.5 wt.-%, most preferably 15±5 wt.-%, and in particular 15±2.5 wt.-%, based on the total weight of the pharmaceutical dosage form.

In still another preferred embodiment, the content of further polymer is within the range of 20±15 wt.-%, more preferably 20±12.5 wt.-%, still more preferably 20±10 wt.-%, yet more preferably 20±7.5 wt.-%, most preferably 20±5 wt.-%, and in particular 20±2.5 wt.-%, based on the total weight of the pharmaceutical dosage form.

In yet another preferred embodiment, the content of further polymer is within the range of 25±20 wt.-%, more preferably 25±15 wt.-%, still more preferably 25±10 wt.-%, most preferably 25±7.5 wt.-%, and in particular 25±5 wt.-%, based on the total weight of the pharmaceutical dosage form.

In a preferred embodiment, the further polymer has a weight average molecular weight (M_{w}) of at least 100,000 g/mol, preferably at least 200,000 g/mol or at least 400,000 g/mol, more preferably in the range of about 500,000 g/mol to about 5,000,000 g/mol, and most preferably in the range of about 600,000 g/mol to about 2,000,000 g/mol. Suitable methods to determine M_{w} are known to a person skilled in the art. For instance, M_{w} can be determined by gel permeation chromatography (GPC).

In a preferred embodiment, the pK_{A} of the further polymer is 6.0±2.0, more preferably 6.0±1.5, even more preferably 6.0±1.0, and most preferably 6.0±0.5. In another preferred embodiment, the pK_{A} of the further polymer is 7.0±2.0, more preferably 7.0±1.5, even more preferably 7.0±1.0, and most preferably 7.0±0.5. In still another preferred embodiment, the pK_{A} of the further polymer is 8.0±2.0, more preferably 8.0±1.5, even more preferably 8.0±1.0, and most preferably 8.0±0.5.

In a preferred embodiment, the pH (in 1 wt % aqueous dispersion) of the further polymer is 3.0±3.0, more preferably 3.0±2.0, even more preferably 3.0±1.5, and most preferably 3.0±1.0.

In another preferred embodiment, the pH (in 1 wt % aqueous dispersion) of the further polymer is 6.0±3.0, more preferably 6.0±2.0, even more preferably 6.0±1.5, and most preferably 6.0±1.0.

The further polymer preferably exhibits a viscosity of 2,000 to 100,000 mPa s (cp), more preferably 3,000 to 80,000 mPa s, still more preferably 4,000 to 60,000 mPa s, measured by means of a Brookfield viscometer (RVF, 20 rpm) in a 0.5 wt.-% aqueous solution at pH 7.5 and 25° C.

In a preferred embodiment, the further polymer exhibits a viscosity of more than 10,000 mPa (cp), preferably at least 11,000 mPa s, more preferably at least 15,000 mPa s, still more preferably at least 20,000 mPa s or at least 30,000 mPa s, measured by means of a Brookfield viscometer (RVF, 20 rpm) in a 0.5 wt.-% aqueous solution at pH 7.5 and 25° C.

In a preferred embodiment the relative weight ratio of said polyalkylene oxide and said further polymer is within the range of from 20:1 to 1:20, more preferably 15:1 to 1:10, still more preferably 10:1 to 1:5, yet more preferably 8:1 to 1:1, most preferably 8:1 to 2:1 and in particular 8:1 to 3:1. In a preferred embodiment, the relative weight ratio of said polyalkylene oxide and said further polymer is within the range of from 10:1 to 5:1, more preferably 8:1 to 5:1, most preferably 7:1 to 5:1. In another preferred embodiment the relative weight ratio of said polyalkylene oxide and said further polymer is within the range of from 20:1 to 1:20, more preferably 15:1 to 1:10, still more preferably 5:1 to 1:2 or 10:1 to 1:1, most preferably 5:1 to 1:1, and in particular 2:1 to 1:1.

In a preferred embodiment, the content of said further polymer amounts to 0.5 to 25 wt.-%, more preferably 1.0 to 20 wt.-%, still more preferably 2.0 to 22.5 wt.-%, yet more preferably 3.0 to 20 wt.-% and most preferably 4.0 to 17.5 wt.-% and in particular 5.0 to 15 wt.-%, based on the total weight of the pharmaceutical dosage form.

In another preferred embodiment, the content of said further polymer amounts to 0.5 to 40 wt.-%, more preferably 1.0 to 35 wt.-%, still more preferably 5.0 to 32.5 wt.-%, yet more preferably 10 to 30 wt.-% and most preferably 12.5 to 27.5 wt.-% and in particular 15 to 25 wt.-%, based on the total weight of the pharmaceutical dosage form.

All polymers are preferably employed as powders. They can be soluble in water.

Preferably, the pharmaceutical dosage form according to the invention is thermoformed, more preferably hot-melt extruded, although also other methods of thermoforming may be used in order to manufacture the pharmaceutical dosage form according to the invention, such as press-molding at elevated temperature or heating of tablets that were manufactured by conventional compression in a first step and then heated above the softening temperature of the polymer in the tablet in a second step to form hard tablets. In this regards, thermoforming means forming or molding of a mass after the application of heat. In a preferred embodiment, the pharmaceutical dosage form is thermoformed by hot-melt extrusion.

In a preferred embodiment, the pharmaceutical dosage form according to the invention has an overall density within the range of 1.19±0.30 g/cm³, more preferably 1.19±0.25 g/cm³, still more preferably 1.19±0.20 g/cm³, yet more preferably 1.19±0.15 g/cm³, most preferably 1.19±0.10 g/cm³, and in particular 1.19±0.05 g/cm³. Preferably, the overall density of the pharmaceutical dosage form according to the invention is 1.17±0.02 g/cm³, 1.19±0.02 g/cm³ or 1.21±0.02 g/cm³. Methods for measuring the density of a pharmaceutical dosage form are known to a person skilled in the art. The overall density of a pharmaceutical dosage form can for example be determined by means of the mercury porosimetry method or the helium pycnometer method as described in Ph. Eur.

In a preferred embodiment, the pharmaceutical dosage form has a total weight within the range of 100±75 mg, more preferably 100±50 mg, most preferably 100±25 mg. In another preferred embodiment, the pharmaceutical dosage form has a total weight within the range of 200±75 mg, more preferably 200±50 mg, most preferably 200±25 mg. In another preferred embodiment, the pharmaceutical dosage form has a total weight within the range of 250±75 mg, more preferably 250±50 mg, most preferably 250±25 mg. In still another preferred embodiment, the pharmaceutical dosage form has a total weight within the range of 300±75 mg, more preferably 300±50 mg, most preferably 300±25 mg. In yet another preferred embodiment, the pharmaceutical dosage form has a total weight within the range of 400±75 mg, more preferably 400±50 mg, most preferably 400±25 mg.

In a preferred embodiment, the pharmaceutical dosage form has a total weight within the range of 500±250 mg, more preferably 500±200 mg, most preferably 500±150 mg. In another preferred embodiment, the pharmaceutical dosage form has a total weight within the range of 750±250 mg, more preferably 750±200 mg, most preferably 750±150 mg. In another preferred embodiment, the pharmaceutical dosage form has a total weight within the range of 1000±250 mg, more preferably 1000±200 mg, most preferably 1000±150 mg. In still another preferred embodiment, the pharmaceutical dosage form has a total weight within the range of 1250±250 mg, more preferably 1250±200 mg, most preferably 1250±150 mg.

The pharmaceutical dosage form according to the invention contains a pharmacologically active compound, preferably a pharmacologically active compound having psychotropic activity, more preferably an opioid. Preferably, the pharmacologically active compound is selected from the group consisting of opiates, opioids, stimulants, tranquilizers, and other narcotics.

For the purpose of the specification, the term pharmacologically active compound also includes the free base and the physiologically acceptable salts thereof.

According to the ATC index, opioids are divided into natural opium alkaloids, phenylpiperidine derivatives, diphenylpropylamine derivatives, benzomorphan derivatives, oripavine derivatives, morphinan derivatives and others. Examples of natural opium alkaloids are morphine, opium, hydromorphone, nicomorphine, oxycodone, dihydrocodeine, diamorphine, papavereturn, and codeine. Further pharmacologically active compounds are, for example, ethylmorphine, hydrocodone, oxymorphone, and the physiologically acceptable derivatives thereof or compounds, preferably the salts and solvates thereof, preferably the hydrochlorides thereof, physiologically acceptable enantiomers, stereoisomers, diastereomers and racemates and the physiologically acceptable derivatives thereof, preferably ethers, esters or amides.

The following opiates, opioids, tranquillizers or other narcotics are substances with a psychotropic action, i.e. have a potential of abuse, and hence are preferably contained in the pharmaceutical dosage form according to the invention: alfentanil, allobarbital, allylprodine, alphaprodine, alprazolam, amfepramone, amphetamine, amphetaminil, amobarbital, anileridine, apocodeine, axomadol, barbital, bemidone, benzylmorphine, bezitramide, bromazepam, brotizolam, buprenorphine, butobarbital, butorphanol, camazepam, carfentanil, cathine/D-norpseudoephedrine, chlordiazepoxide, clobazam clofedanol, clonazepam, clonitazene, clorazepate, clotiazepam, cloxazolam, cocaine, codeine, cyclobarbital, cyclorphan, cyprenorphine, delorazepam, desomorphine, dextromoramide, dextropropoxyphene, dezocine, diampromide, diamorphone, diazepam, dihydrocodeine, dihydromorphine, dihydromorphone, dimenoxadol, dimephetamol, dimethylthiambutene, dioxaphetylbutyrate, dipipanone, dronabinol, eptazocine, estazolam, ethoheptazine, ethylmethylthiambutene, ethyl loflazepate, ethylmorphine, etonitazene, etorphine, faxeladol, fencamfamine, fenethylline, fenpipramide, fenproporex, fentanyl, fludiazepam, flunitrazepam, flurazepam, halazepam, haloxazolam, heroin, hydrocodone, hydromorphone, hydroxypethidine, isomethadone, hydroxymethylmorphinan, ketazolam, ketobemidone, levacetylmethadol (LAAM), levomethadone, levorphanol, levophenacylmorphane, levoxemacin, lisdexamfetamine dimesylate, lofentanil, loprazolam, lorazepam, lormetazepam, mazindol, medazepam, mefenorex, meperidine, meprobamate, metapon, meptazinol, metazocine, methylmorphine, metamphetamine, methadone, methaqualone, 3-methylfentanyl, 4-methylfentanyl, methylphenidate, methylphenobarbital, methyprylon, metopon, midazolam, modafinil, morphine, myrophine, nabilone, nalbuphene, nalorphine, narceine, nicomorphine, nimetazepam, nitrazepam, nordazepam, norlevorphanol, normethadone, normorphine, norpipanone, opium, oxazepam, oxazolam, oxycodone, oxymorphone, Papaver somniferum, papavereturn, pernoline, pentazocine, pentobarbital, pethidine, phenadoxone, phenomorphane, phenazocine, phenoperidine, piminodine, pholcodeine, phenmetrazine, phenobarbital, phentermine, pinazepam, pipradrol, piritramide, prazepam, profadol, proheptazine, promedol, properidine, propoxyphene, remifentanil, secbutabarbital, secobarbital, sufentanil, tapentadol, temazepam, tetrazepam, tilidine (cis and trans), tramadol, triazolam, vinylbital, N-(1-methyl-2-piperidinoethyl)-N-(2-pyridyl)propionamide, (1R,2R)-3-(3-dimethylamino-1-ethyl-2-methyl-propyl)phenol, (1R,2R,4S)-2-(dimethylamino)methyl-4-(p-fluorobenzyloxy)-1-(m-methoxyphenyl)cyclohexanol, (1R,2R)-3-(2-dimethylaminomethyl-cyclohexyl)phenol, (1S,2S)-3-(3-dimethylamino-1-ethyl-2-methyl-propyl)phenol, (2R,3R)-1-dimethylamino-3(3-methoxyphenyl)-2-methyl-pentan-3-ol, (1RS,3RS,6RS)-6-dimethylaminomethyl-1-(3-methoxyphenyl)-cyclohexane-1,3-diol, preferably as racemate, 3-(2-dimethylaminomethyl-1-hydroxy-cyclohexyl)phenyl 2-(4-isobutyl-phenyl)propionate, 3-(2-dimethylaminomethyl-1-hydroxy-cyclohexyl)phenyl 2-(6-methoxy-naphthalen-2-yl)propionate, 3-(2-dimethylaminomethyl-cyclohex-1-enyl)-phenyl 2-(4-isobutyl-phenyl)propionate, 3-(2-dimethylaminomethyl-cyclohex-1-enyl)-phenyl 2-(6-methoxy-naphthalen-2-yl)propionate, (RR—SS)-2-acetoxy-4-trifluoromethyl-benzoic acid 3-(2-dimethylaminomethyl-1-hydroxy-cyclohexyl)-phenyl ester, (RR—SS)-2-hydroxy-4-trifluoromethyl-benzoic acid 3-(2-dimethylaminomethyl-1-hydroxy-cyclohexyl)-phenyl ester, (RR—SS)-4-chloro-2-hydroxy-benzoic acid 3-(2-dimethylaminomethyl-1-hydroxy-cyclohexyl)-phenyl ester, (RR—SS)-2-hydroxy-4-methyl-benzoic acid 3-(2-dimethylaminomethyl-1-hydroxy-cyclohexyl)-phenyl ester, (RR—SS)-2-hydroxy-4-methoxy-benzoic acid 3-(2-dimethylaminomethyl-1-hydroxy-cyclohexyl)-phenyl ester, (RR—SS)-2-hydroxy-5-nitro-benzoic acid 3-(2-dimethylaminomethyl-1-hydroxy-cyclohexyl)-phenyl ester, (RR—SS)-2′,4′-difluoro-3-hydroxy-biphenyl-4-carboxylic acid 3-(2-dimethylaminomethyl-1-hydroxy-cyclohexyl)-phenyl ester, and corresponding stereoisomers, in each case the corresponding derivatives thereof, physiologically acceptable enantiomers, stereoisomers, diastereomers and racemates and the physiologically acceptable derivatives thereof, e.g. ethers, esters or amides, and in each case the physiologically acceptable compounds thereof, in particular the acid or base addition salts thereof and solvates, e.g. hydrochlorides.

In a preferred embodiment the pharmaceutical dosage form according to the invention contains an opioid selected from the group consisting of DPI-125, M6G (CE-04-410), ADL-5859, CR-665, NRP290 and sebacoyl dinalbuphine ester.

Particularly preferred pharmacologically active compounds include hydromorphone, oxymorphone, oxycodone, tapentadol, and the physiologically acceptable salts thereof. In a preferred embodiment the pharmaceutical dosage form according to the invention contains one pharmacologically active compound or more pharmacologically active compounds selected from the group consisting of oxymorphone, hydromorphone and morphine. In another preferred embodiment, the pharmacologically active compound is selected from the group consisting of tapentadol, faxeladol and axomadol.

In still another preferred embodiment, the pharmacologically active compound is selected from the group consisting of 1,1-(3-dimethylamino-3-phenylpentamethylene)-6-fluoro-1,3,4,9-tetrahydropyrano[3,4-b]indole, particularly its hemicitrate; 1,1-[3-dimethylamino-3-(2-thienyl)-pentamethylene]-1,3,4,9-tetrahydropyrano[3,4-b]indole, particularly its citrate; and 1,1-[3-dimethylamino-3-(2-thienyl)pentamethylene]-1,3,4,9-tetrahydropyrano[3,4-b]-6-fluoroindole, particularly its hemicitrate. These compounds are known from, e.g., WO 2004/043967, WO 2005/066183.

The pharmacologically active compound may be present in form of a physiologically acceptable salt, e.g. physiologically acceptable acid addition salt.

Physiologically acceptable acid addition salts comprise the acid addition salt forms which can conveniently be obtained by treating the base form of the active ingredient with appropriate organic and inorganic acids. Active ingredients containing an acidic proton may be converted into their non-toxic metal or amine addition salt forms by treatment with appropriate organic and inorganic bases. The term addition salt also comprises the hydrates and solvent addition forms which the active ingredients are able to form. Examples of such forms are e.g. hydrates, alcoholates and the like.

The content of the pharmacologically active compound in the pharmaceutical dosage form is not limited. The pharmacologically active compound is present in the pharmaceutical dosage form in a therapeutically effective amount. The amount that constitutes a therapeutically effective amount varies according to the active ingredients being used, the condition being treated, the severity of said condition, the patient being treated, and whether the pharmaceutical dosage form is designed for an immediate or retarded release.

Preferably, the content of the pharmacologically active compound is within the range of from 0.01 to 80 wt.-%, more preferably 0.1 to 50 wt.-%, still more preferably 1 to 25 wt.-%, based on the total weight of the pharmaceutical dosage form. In a preferred embodiment, the content of pharmacologically active compound is within the range of from 7±6 wt.-%, more preferably 7±5 wt.-%, still more preferably 5±4 wt.-%, 7±4 wt.-% or 9±4 wt.-%, most preferably 5±3 wt.-%, 7±3 wt.-% or 9±3 wt.-%, and in particular 5±2 wt.-%, 7±2 wt.-% or 9±2 wt.-%, based on the total weight of the pharmaceutical dosage form. In another preferred embodiment, the content of pharmacologically active compound is within the range of from 11±10 wt.-%, more preferably 11±9 wt.-%, still more preferably 9±6 wt.-%, 11±6 wt.-%, 13±6 wt.-% or 15±6 wt.-%, most preferably 11±4 wt.-%, 13±4 wt.-% or 15±4 wt.-%, and in particular 11±2 wt.-%, 13±2 wt.-% or 15±2 wt.-%, based on the total weight of the pharmaceutical dosage form. In a further preferred embodiment, the content of pharmacologically active compound is within the range of from 20±6 wt.-%, more preferably 20±5 wt.-%, still more preferably 20±4 wt.-%, most preferably 20±3 wt.-%, and in particular 20±2 wt.-%, based on the total weight of the pharmaceutical dosage form.

Preferably, the total amount of the pharmacologically active compound that is contained in the pharmaceutical dosage form is within the range of from 0.01 to 200 mg, more preferably 0.1 to 190 mg, still more preferably 1.0 to 180 mg, yet more preferably 1.5 to 160 mg, most preferably 2.0 to 100 mg and in particular 2.5 to 80 mg.

In a preferred embodiment, the pharmacologically active compound is contained in the pharmaceutical dosage form in an amount of 7.5±5 mg, 10±5 mg, 20±5 mg, 30±5 mg, 40±5 mg, 50±5 mg, 60±5 mg, 70±5 mg, 80±5 mg, 90±5 mg, 100±5 mg, 110±5 mg, 120±5 mg, 130±5, 140±5 mg, 150±5 mg, or 160±5 mg. In another preferred embodiment, the pharmacologically active compound is contained in the pharmaceutical dosage form in an amount of 5±2.5 mg, 7.5±2.5 mg, 10±2.5 mg, 15±2.5 mg, 20±2.5 mg, 25±2.5 mg, 30±2.5 mg, 35±2.5 mg, 40±2.5 mg, 45±2.5 mg, 50±2.5 mg, 55±2.5 mg, 60±2.5 mg, 65±2.5 mg, 70±2.5 mg, 75±2.5 mg, 80±2.5 mg, 85±2.5 mg, 90±2.5 mg, 95±2.5 mg, 100±2.5 mg, 105±2.5 mg, 110±2.5 mg, 115±2.5 mg, 120±2.5 mg, 125±2.5 mg, 130±2.5 mg, 135±2.5 mg, 140±2.5 mg, 145±2.5 mg, 150±2.5 mg, 155±2.5 mg, or 160±2.5 mg.

In a preferred embodiment, pharmacologically active compound is oxymorphone, preferably its hydrochloride salt, and the pharmaceutical dosage form is adapted for administration twice daily. In this embodiment, pharmacologically active compound is preferably contained in the pharmaceutical dosage form in an amount of from 5 to 40 mg. In another particularly preferred embodiment, the pharmacologically active compound is oxymorphone, preferably its hydrochloride salt, and the pharmaceutical dosage form is adapted for administration once daily. In this embodiment, pharmacologically active compound is preferably contained in the pharmaceutical dosage form in an amount of from 10 to 80 mg.

In another preferred embodiment, pharmacologically active compound is oxycodone, preferably its hydrochloride salt, and the pharmaceutical dosage form is adapted for administration twice daily. In this embodiment, pharmacologically active compound is preferably contained in the pharmaceutical dosage form in an amount of from 5 to 80 mg, preferably 5 mg, 10 mg, 20 mg or 40 mg. In another particularly preferred embodiment, the pharmacologically active compound is oxycodone, preferably its hydrochloride salt, and the pharmaceutical dosage form is adapted for administration once daily. In this embodiment, pharmacologically active compound is preferably contained in the pharmaceutical dosage form in an amount of from 10 to 320 mg.

In still another particularly preferred embodiment, pharmacologically active compound is hydromorphone, preferably its hydrochloride, and the pharmaceutical dosage form is adapted for administration twice daily. In this embodiment, pharmacologically active compound is preferably contained in the pharmaceutical dosage form in an amount of from 2 to 52 mg. In another particularly preferred embodiment, pharmacologically active compound is hydromorphone, preferably its hydrochloride salt, and the pharmaceutical dosage form is adapted for administration once daily. In this embodiment, pharmacologically active compound is preferably contained in the pharmaceutical dosage form in an amount of from 4 to 104 mg.

In yet another particularly preferred embodiment, pharmacologically active compound is tapentadol, preferably its hydrochloride, and the pharmaceutical dosage form is adapted for administration twice daily. In this embodiment, pharmacologically active compound is preferably contained in the pharmaceutical dosage form in an amount of from 25 to 250 mg. In another particularly preferred embodiment, the pharmacologically active compound is tapentadol, preferably its hydrochloride salt, and the pharmaceutical dosage form is adapted for administration once daily. In this embodiment, pharmacologically active compound is preferably contained in the pharmaceutical dosage form in an amount of from 50 to 600 mg.

The pharmaceutical dosage form according to the invention is characterized by excellent storage stability. Preferably, after storage for 4 weeks at 40° C. and 75% rel. humidity, the content of pharmacologically active compound amounts to at least 90%, more preferably at least 91%, still more preferably at least 92%, yet more preferably at least 93%, most preferably at least 94% and in particular at least 95%, of its original content before storage.

Suitable methods for measuring the content of the pharmacologically active compound in the pharmaceutical dosage form are known to the skilled artisan. In this regard it is referred to the Eur. Ph. or the USP, especially to reversed phase HPLC analysis. Preferably, the pharmaceutical dosage form is stored in closed, preferably sealed containers, most preferably being equipped with an oxygen scavenger, in particular with an oxygen scavenger that is effective even at low relative humidity.

In a preferred embodiment, after oral administration of the pharmaceutical dosage form according to the invention, in vivo the average peak plasma level (Cₘₐₓ) of the pharmacologically active compound is on average reached after tₘₐₓ 4.0±2.5 h, more preferably after tₘₐₓ 4.0±2.0 h, still more preferably after tₘₐₓ 4.0±1.5 h, most preferably after tₘₐₓ 4.0±1.0 h and in particular after tₘₐₓ 4.0±0.5 h. In another preferred embodiment, after oral administration of the pharmaceutical dosage form according to the invention, in vivo the average peak plasma level (Cₘₐₓ) of the pharmacologically active compound is on average reached after tₘₐₓ 5.0±2.5 h, more preferably after tₘₐₓ 5.0±2.0 h, still more preferably after tₘₐₓ 5.0±1.5 h, most preferably after tₘₐₓ 5.0±1.0 h and in particular after tₘₐₓ 5.0±0.5 h. In still another preferred embodiment, after oral administration of the pharmaceutical dosage form according to the invention, in vivo the average peak plasma level (Cₘₐₓ) of the pharmacologically active compound is on average reached after tₘₐₓ 6.0±2.5 h, more preferably after tₘₐₓ 6.0±2.0 h, still more preferably after tₘₐₓ 6.0±1.5 h, most preferably after tₘₐₓ 6.0±1.0 h and in particular after tₘₐₓ 6.0±0.5 h.

In a preferred embodiment, the average value for t_{1/2} of the pharmacologically active compound after oral administration of the pharmaceutical dosage form according to the invention in vivo is 4.0±2.5 h, more preferably 4.0±2.0 h, still more preferably 4.0±1.5 h, most preferably 4.0±1.0 h, and in particular 4.0±0.5 h. In another preferred embodiment, the average value for t_{1/2} of the pharmacologically active compound after oral administration of the pharmaceutical dosage form according to the invention in vivo is preferably 5.0±2.5 h, more preferably 5.0±2.0 h, still more preferably 5.0±1.5 h, most preferably 5.0±1.0 h, and in particular 5.0±0.5 h. In still another preferred embodiment, the average value for t_{1/2} of the pharmacologically active compound after oral administration of the pharmaceutical dosage form according to the invention in vivo is preferably 6.0±2.5 h, more preferably 6.0±2.0 h, still more preferably 6.0±1.5 h, most preferably 6.0±1.0 h, and in particular 6.0±0.5 h.

Preferably, Cₘₐₓ of the pharmacologically active compound does not exceed 0.01 ng/ml, or 0.05 ng/ml, or 0.1 ng/ml, or 0.5 ng/ml, or 1.0 ng/ml, or 2.5 ng/ml, or 5 ng/ml, or 10 ng/ml, or ng/ml, or 30 ng/ml, or 40 ng/ml, or 50 ng/ml, or 75 ng/ml, or 100 ng/ml, or 150 ng/ml, or 200 ng/ml, or 250 ng/ml, or 300 ng/ml, or 350 ng/ml, or 400 ng/ml, or 450 ng/ml, or 500 ng/ml, or 750 ng/ml, or 1000 ng/ml.

The pharmaceutical dosage form according to the invention contains at least one non-ionic surfactant.

In a preferred embodiment, the nonionic surfactant has a hydrophilic-lipophilic balance (HLB) of at least 10, preferably at least 12, more preferably at least 14, still more preferably at least 16, yet more preferably at least 18, even more preferably at least 20, most preferably at least 22, and in particular at least or more than 24.

The hydrophilic-lipophilic balance (HLB value) can be estimated according to Griffin's method (Griffin, W. C., J. Soc. Cosmet. Chem. 1 (1949) 311).

Preferably, however, the HLB value is calculated by the incremental method, i.e. by adding the individual HLB increments of all hydrophobic and hydrophilic groups present in the molecule. HLB increments of many hydrophobic and hydrophilic groups can be found, e.g., in Fiedler, H. P., Encyclopedia of Excipients, Editio Cantor Verlag, Aulendorf, 6th Edition, 2007. The HLB value can further be determined experimentally, e.g. by partition chromatography or HPLC.

In another preferred embodiment, the nonionic surfactant exhibits a surface tension in 0.1% aqueous solution at 25° C. of at least 35 dynes/cm, more preferably at least 40 dynes/cm, still more preferably at least 43 dynes/cm, yet more preferably at least 45 dynes/cm, even more preferably at least 47 dynes/cm, in particular at least 50 dynes/cm.

In another preferred embodiment, the nonionic surfactant exhibits a viscosity of at most 4000 mPa·s, more preferably at most 3500 mPa·s, still more preferably at most 3000 mPa·s, yet more preferably at most 2500 mPa·s, even more preferably at most 2000 mPa·s, most preferably at most 1500 mPa·s, and in particular at most 1000 mPa·s, measured at 70° C. using a model LVF or LVT Brookfield viscosimeter.

Suitable non-ionic surfactants include but are not limited to
polyoxypropylene-polyoxyethylene block copolymers (e.g., poloxamers or poloxamines), preferably according to general formula (I-a)

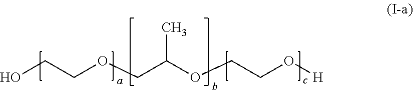

wherein a and c are each independently an integer of from 5 to 250, and b is an integer of from 10 to 100; preferably, a=c≠b; and/or a=c>b;
or according to general formula (I-b)

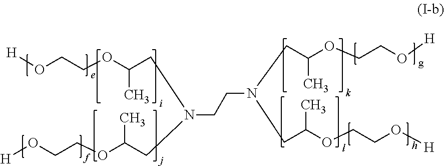

wherein e, f, g and h are each independently an integer of from 1 to 150, and i, j, k and l are each independently an integer of from 2 to 50; and preferably, the ratio (e+f+g+h)/(i+j+k+l) is an integer of from 0.015 to 30;
fatty alcohols that may be linear or branched, such as cetylalcohol, stearylalcohol, cetylstearyl alcohol, 2-octyldodecane-1-ol and 2-hexyldecane-1-ol;
sterols, such as cholesterole;
partial fatty acid esters of sorbitan such as sorbitanmonolaurate, sorbitanmonopalmitate, sorbitanmonostearate, sorbitantristearate, sorbitanmonooleate, sorbitansesquioleate and sorbitantrioleate;
partial fatty acid esters of polyoxyethylene sorbitan (polyoxyethylene-sorbitan-fatty acid esters), preferably a fatty acid monoester of polyoxyethylene sorbitan, a fatty acid diester of polyoxyethylene sorbitan, or a fatty acid triester of polyoxyethylene sorbitan; e.g. mono- and tri-lauryl, palmityl, stearyl and oleyl esters, such as the type known under the name “polysorbat” and commercially available under the trade name “Tween” including Tween® 20 [polyoxyethylene(20)sorbitan monolaurate], Tween® 21 [polyoxyethylene(4)sorbitan monolaurate], Tween® 40 [polyoxyethylene(20)sorbitan monopalmitate], Tween® 60 [polyoxyethylene(20)sorbitan monostearate], Tween® 65 [polyoxyethylene(20)sorbitan tristearate], Tween® 80 [polyoxyethylene(20)sorbitan monooleate], Tween 81 [polyoxyethylene(5)sorbitan monooleate], and Tween® 85 [polyoxyethylene(20)sorbitan trioleate]; preferably a fatty acid monoester of polyoxyethylenesorbitan according to general formula (I-c)

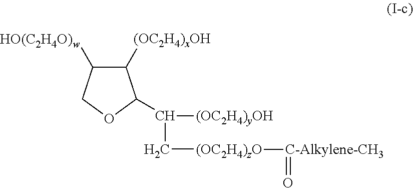

wherein (w+x+y+z) is within the range of from 15 to 100, preferably 16 to 80, more preferably 17 to 60, still more preferably 18 to 40 and most preferably 19 to 21; and alkylene is an optionally unsaturated alkylene group comprising 6 to 30 carbon atoms, more preferably 8 to 24 carbon atoms and most preferably 10 to 16 carbon atoms;
polyoxyethyleneglycerole fatty acid esters such as mixtures of mono-, di- and triesters of glycerol and di- and monoesters of macrogols having molecular weights within the range of from 200 to 4000 g/mol, e.g., macrogolglycerolcaprylocaprate, macrogolglycerollaurate, macrogolglycerolococoate, macrogolglycerollinoleate, macrogol-20-glycerolmonostearate, macrogol-6-glycerolcaprylocaprate, macrogolglycerololeate; macrogolglycerolstearate, macrogolglycerolhydroxystearate (e.g. Cremophor® RH 40), and macrogolglycerolrizinoleate (e.g. Cremophor® EL);
polyoxyethylene fatty acid esters, the fatty acid preferably having from about 8 to about 18 carbon atoms, e.g. macrogololeate, macrogolstearate, macrogol-15-hydroxystearate, polyoxyethylene esters of 12-hydroxystearic acid, such as the type known and commercially available under the trade name “Solutol HS 15”; preferably according to general formula (I-d)

CH₃CH₂—(OCH₂CH₃)ₙ—O—CO—(CH₂)ₘCH₃ (I-d)
wherein n is an integer of from 6 to 500, preferably 7 to 250, more preferably 8 to 100, still more preferably 9 to 75, yet more preferably 10 to 50, even more preferably 11 to 30, most preferably 12 to 25, and in particular 13 to 20; and
wherein m is an integer of from 6 to 28; more preferably 6 to 26, still more preferably 8 to 24, yet more preferably 10 to 22, even more preferably 12 to 20, most preferably 14 to 18 and in particular 16;
polyoxyethylene fatty alcohol ethers, e.g. macrogolcetylstearylether, macrogollaurylether, macrogololeylether, macrogolstearylether;
fatty acid esters of saccharose; e.g. saccharose distearate, saccharose dioleate, saccharose dipalmitate, saccharose monostearate, saccharose monooleate, saccharose monopalmitate, saccharose monomyristate and saccharose monolaurate;
fatty acid esters of polyglycerol, e.g. polyglycerololeate; polyoxyethylene esters of alpha-tocopheryl succinate, e.g. D-alpha-tocopheryl-PEG-1000-succinate (TPGS);
polyglycolyzed glycerides, such as the types known and commercially available under the trade names “Gelucire 44/14”, “Gelucire 50/13 and “Labrasol”;
reaction products of a natural or hydrogenated castor oil and ethylene oxide such as the various liquid surfactants known and commercially available under the trade name “Cremophor”; and
partial fatty acid esters of multifunctional alcohols, such as glycerol fatty acid esters, e.g. mono- and tri-lauryl, palmityl, stearyl and ° leyl esters, for example glycerol monostearate, glycerol monooleate, e.g. glyceryl monooleate 40, known and commercially available under the trade name “Peceol”; glycerole dibehenate, glycerole distearate, glycerole monolinoleate; ethyleneglycol monostearate, ethyleneglycol monopalmitostearate, pentaerythritol monostearate.

In a preferred embodiment, the nonionic surfactant is a thermosensitive polymer, in particular an inverse thermosensitive polymer, i.e. a polymer that is soluble in water at a comparatively low temperature, e.g. below or about 20° C., but gels (forms a gel) at higher temperatures, e.g. above 35° C.

For the purpose of the specification, an “inverse thermosensitive polymer” preferably is a polymer exhibiting an atypical dependency of viscosity from temperature; while aqueous dispersions of conventional polymers typically show decreased viscosities at increased temperatures, the viscosity of an aqueous dispersion of an inverse thermosensitive polymer according to the invention increases at increased temperatures, at least within a certain temperature range above ambient temperature. Preferably, the increase of viscosity that is induced by an increase of temperature leads to gel formation so that an aqueous dispersion of an inverse thermosensitive polymer according to the invention preferably forms a liquid solution at ambient temperature but a viscous gel at elevated temperature. Polymeric nonionic surfactants exhibiting these properties are known to the skilled artisan.

A skilled person recognizes that viscosity and gel strength may decrease again, once a certain temperature is exceeded. Thus, an aqueous dispersion of an inverse thermosensitive polymer according to the invention preferably has a viscosity maximum, which at a concentration of 25 wt.-%, relative to the total weight of the aqueous dispersion, is preferably within the range 45±20° C., or 55±20° C., or 65±20° C., or 75±20° C.

Thus, the nonionic surfactant according to the invention preferably forms a liquid solution in water at ambient temperature, and when the temperature is increased, the surfactant forms an aqueous gel, at least within a certain temperature range above ambient temperature.

Preferably, in pure water at a concentration of 25 wt.-% the nonionic surfactant forms an aqueous dispersion having a viscosity η₁ at a temperature T₁ of 20° C. and a viscosity η₂ at a temperature T₂ of more than 20° C. (i.e. T₂>T₁), where η₂>η₁. This does not necessarily mean that viscosity η₂ at any temperature T₂ above 20° C. must be greater than viscosity η₁ at 20° C. Instead, this means that there is at least one temperature T₂ above 20° C. at which viscosity η₂ of the aqueous dispersion is greater than viscosity η₁ at T₁ (=20° C.).

Preferably, an aqueous solution comprising at least 20 wt.-% or at least 25 wt.-% nonionic surfactant shows a thermoreversible behavior, i.e. the viscosity of the solution increases with increasing temperature and decreases with decreasing temperature, and repeated heating and cooling does not affect this property. Preferably, the aqueous solution exhibits a thermoreversible behavior with a maximum viscosity between 30 and 80° C.

In an especially preferred embodiment, the aqueous dispersion of the nonionic surfactant is a liquid at 20° C. and forms a semi-solid gel upon heating to a temperature of at most 80° C., more preferably 60° C., most preferably at most 45° C., and in particular at most 37° C.

Preferably, the sol-gel transition temperature, i.e. the temperature at which the phase transition occurs, is within the range of from 10° C. to 80° C., more preferably within the range of from 15° C. to 75° C., and most preferably within the range of from 20° C. to 60° C.

For example, various poloxamines and poloxamers, including poloxamer 407 and poloxamer 188, show inverse thermosensitivity.

Particularly preferably, the nonionic surfactant is a polyoxypropylene-polyoxyethylene block copolymer, preferably selected from poloxamers and poloxamines, in particular poloxamers according to general formula (I-a) and poloxamines according to general formula (I-b).

In a particular preferred embodiment, the nonionic surfactant is a polyoxypropylene-polyoxyethylene block copolymer according to general formula (I-a)

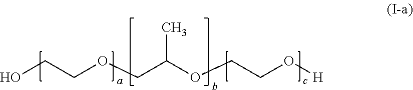

wherein a and c are each independently an integer of from 5 to 250, and b is an integer of from 10 to 100; and preferably, a=c≠b; and/or a=c>b. More preferably, a and c are each independently an integer of from 10 to 120, and b is an integer of from 15 to 75; and preferably, a=c>b. Polyoxypropylene-polyoxyethylene block copolymers of this type are also known as poloxamers and are commercially available under the trade name Pluronics.

In a preferred embodiment, a, b and c are each independently an integer as specified as preferred embodiments N¹ to N³² in the table here below:

| | a | B | c | | a | b | c |
|---|---|---|---|---|---|---|---|
| N¹ | 80 ± 75 | 27 ± 17 | 80 ± 75 | N⁹ | 80 ± 27 | 27 ± 9 | 80 ± 27 |
| N² | 80 ± 65 | 27 ± 16 | 80 ± 65 | N¹⁰ | 80 ± 23 | 27 ± 8 | 80 ± 23 |
| N³ | 80 ± 55 | 27 ± 15 | 80 ± 55 | N¹¹ | 80 ± 19 | 27 ± 7 | 80 ± 19 |
| N⁴ | 80 ± 50 | 27 ± 14 | 80 ± 50 | N¹² | 80 ± 15 | 27 ± 6 | 80 ± 15 |
| N⁵ | 80 ± 45 | 27 ± 13 | 80 ± 45 | N¹³ | 80 ± 12 | 27 ± 5 | 80 ± 12 |
| N⁶ | 80 ± 40 | 27 ± 12 | 80 ± 40 | N¹⁴ | 80 ± 9 | 27 ± 4 | 80 ± 9 |
| N⁷ | 80 ± 35 | 27 ± 11 | 80 ± 35 | N¹⁵ | 80 ± 6 | 27 ± 3 | 80 ± 6 |
| N⁸ | 80 ± 31 | 27 ± 10 | 80 ± 31 | N¹⁶ | 80 ± 3 | 27 ± 2 | 80 ± 3 |
| N¹⁷ | 12 ± 11 | 20 ± 15 | 12 ± 11 | N²⁵ | 101 ± 80 | 56 ± 35 | 101 ± 80 |
| N¹⁸ | 12 ± 8 | 20 ± 12 | 12 ± 8 | N²⁶ | 101 ± 55 | 56 ± 21 | 101 ± 55 |
| N¹⁹ | 12 ± 5 | 20 ± 8 | 12 ± 5 | N²⁷ | 101 ± 31 | 56 ± 12 | 101 ± 31 |
| N²⁰ | 12 ± 2 | 20 ± 4 | 12 ± 2 | N²⁸ | 101 ± 15 | 56 ± 8 | 101 ± 15 |
| N²¹ | 64 ± 45 | 37 ± 13 | 64 ± 45 | N²⁹ | 141 ± 120 | 44 ± 31 | 141 ± 120 |
| N²² | 64 ± 20 | 37 ± 10 | 64 ± 20 | N³⁰ | 141 ± 90 | 44 ± 27 | 141 ± 90 |
| N²³ | 64 ± 12 | 37 ± 7 | 64 ± 12 | N³¹ | 141 ± 35 | 44 ± 19 | 141 ± 35 |
| N²⁴ | 64 ± 5 | 37 ± 5 | 64 ± 5 | N³² | 141 ± 17 | 44 ± 11 | 141 ± 17 |

In another preferred embodiment, the nonionic surfactant is a polyoxypropylene-polyoxyethylene block copolymer according to general formula (I-b)

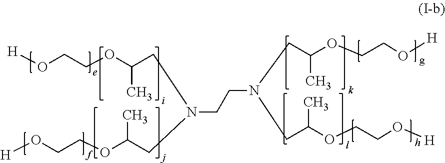

wherein e, f, g and h are each independently an integer of from 1 to 150, and i, j, k and l are each independently an integer of from 2 to 50; and preferably, the ratio (e+f+g+h)/(i+j+k+l) is from 0.015 to 30, in particular from 1 to 10. More preferably, e, f, g and h are each independently an integer of from 3 to 50, and i, j, k and l are each independently an integer of from 2 to 30. Tetrafunctional polyoxypropylene-polyoxyethylene block copolymers of this type are also known as poloxamines and are commercially available under the trade name Tetronics.

Preferably, the nonionic surfactant, preferably according to general formula (I-a) or according to general formula (I-b) has an average molecular weight of at least 2,000 g/mol, more preferably at least 3,000 g/mol, still more preferably at least 4,000 g/mol, yet more preferably at least 5,000 g/mol, even more preferably at least 6,000 g/mol, most preferably at least 7,000 g/mol, and in particular at least 7,500 g/mol.

Preferably, the nonionic surfactant, preferably according to general formula (I-a) or according to general formula (I-b) has an average molecular weight of at most 30,000 g/mol, more preferably at most 25,000 g/mol, still more preferably at most 20,000 g/mol, yet more preferably at most 15,000 g/mol, even more preferably at most 12,500 g/mol, most preferably at most 10,000 g/mol, and in particular at most 9,500 g/mol.

Preferably, the nonionic surfactant, preferably according to general formula (I-a) or according to general formula (I-b) has an average molecular weight as specified as preferred embodiments O¹ to O³² in the table here below:

| g/mol | M_{w} |
|---|---|
| O¹ | 8.600 ± 7.500 |
| O² | 8.600 ± 5.000 |
| O³ | 8.600 ± 4.000 |
| O⁴ | 8.600 ± 3.000 |
| O⁵ | 8.600 ± 2.500 |
| O⁶ | 8.600 ± 2.250 |
| O⁷ | 8.600 ± 2.000 |
| O⁸ | 8.600 ± 1.750 |
| O⁹ | 8.600 ± 1.500 |
| O¹⁰ | 8.600 ± 1.400 |
| O¹¹ | 8.600 ± 1.300 |
| O¹² | 8.600 ± 1.200 |
| O¹³ | 8.600 ± 1.100 |
| O¹⁴ | 8.600 ± 1.000 |
| O¹⁵ | 8.600 ± 950 |
| O¹⁶ | 8.600 ± 920 |
| O¹⁷ | 2.200 ± 1.000 |
| O¹⁸ | 2.200 ± 500 |
| O¹⁹ | 2.200 ± 250 |
| O²⁰ | 7.800 ± 6.000 |
| O²¹ | 7.800 ± 4.000 |
| O²² | 7.800 ± 1.500 |
| O²³ | 7.800 ± 1.000 |
| O²⁴ | 7.800 ± 800 |
| O²⁵ | 12.200 ± 8.000 |
| O²⁶ | 12.200 ± 4.000 |
| O²⁷ | 12.200 ± 3.000 |
| O²⁸ | 12.200 ± 1.500 |
| O²⁹ | 15.000 ± 7.500 |
| O³⁰ | 15.000 ± 5.000 |
| O³¹ | 15.000 ± 3.000 |
| O³² | 15.000 ± 2.000 |

Preferably, the nonionic surfactant, preferably according to general formula (I-a) or according to general formula (I-b) has an oxyethylene content, as determined according to USP or Ph. Eur., of at least 60%, more preferably at least 70%, still more preferably at least 72%, yet more preferably at least 74%, even more preferably at least 76%, most preferably at least 78%, and in particular at least 80%.

Preferably, the nonionic surfactant, preferably according to general formula (I-a) or according to general formula (I-b) has an oxyethylene content, as determined according to USP or Ph. Eur., of at most 90%, more preferably at most 89%, still more preferably at most 88%, yet more preferably at most 87%, even more preferably at most 86%, most preferably at most 85%, and in particular at most 84%.

Preferably, the nonionic surfactant, preferably according to general formula (I-a) or according to general formula (I-b) has an oxyethylene content, as determined according to USP or Ph. Eur., as specified as preferred embodiments P¹ to P³² in the table here below:

| % | OE-content |
|---|---|
| P¹ | 81.8 ± 17.0 |
| P² | 81.8 ± 16.0 |
| P³ | 81.8 ± 15.0 |
| P⁴ | 81.8 ± 14.0 |
| P⁵ | 81.8 ± 13.0 |
| P⁶ | 81.8 ± 12.0 |
| P⁷ | 81.8 ± 11.0 |
| P⁸ | 81.8 ± 10.0 |
| P⁹ | 81.8 ± 9.0 |
| P¹⁰ | 81.8 ± 8.0 |
| P¹¹ | 81.8 ± 7.0 |
| P¹² | 81.8 ± 6.0 |
| P¹³ | 81.8 ± 5.0 |
| P¹⁴ | 81.8 ± 4.0 |
| P¹⁵ | 81.8 ± 3.0 |
| P¹⁶ | 81.8 ± 2.0 |
| P¹⁷ | 46.5 ± 15.0 |
| P¹⁸ | 46.5 ± 10.0 |
| P¹⁹ | 46.5 ± 5.0 |
| P²⁰ | 60.0 ± 20.0 |
| P²¹ | 60.0 ± 15.0 |
| P²² | 70.0 ± 10.0 |
| P²³ | 70.0 ± 8.0 |
| P²⁴ | 70.0 ± 5.0 |
| P²⁵ | 73.0 ± 6.0 |
| P²⁶ | 73.0 ± 4.0 |
| P²⁷ | 75.0 ± 5.0 |
| P²⁸ | 75.0 ± 4.0 |
| P²⁹ | 75.0 ± 3.0 |
| P³⁰ | 85.0 ± 5.0 |
| P³¹ | 85.0 ± 4.0 |
| P³² | 85.0 ± 3.0 |

The content of the nonionic surfactant in the pharmaceutical dosage form is not limited.

Preferably, the content of the nonionic surfactant in the pharmaceutical dosage form according to the invention is such that liquid extraction of the pharmacologically active compound and thus, parenteral administration of the liquid extract, is impeded.

Preferably, the content of the nonionic surfactant is within the range of from 0.01 to 50 wt.-%, more preferably 0.1 to 30 wt.-%, still more preferably 1 to 25 wt.-%, based on the total weight of the pharmaceutical dosage form. In a preferred embodiment, the content of nonionic surfactant is within the range of from 7±6 wt.-%, more preferably 7±5 wt.-%, still more preferably 5±4 wt.-%, 7±4 wt.-% or 9±4 wt.-%, most preferably 5±3 wt.-%, 7±3 wt.-% or 9±3 wt.-%, and in particular 5±2 wt.-%, 7±2 wt.-% or 9±2 wt.-%, based on the total weight of the pharmaceutical dosage form. In another preferred embodiment, the content of nonionic surfactant is within the range of from 11±10 wt.-%, or 13±10 wt.-%, or 15±10 wt.-%, or more preferably 11±9 wt.-%, or 13±9 wt.-%, or 15±9 wt.-%, still more preferably 9±6 wt.-%, 11±6 wt.-%, 13±6 wt.-% or 15±6 wt.-%, most preferably 11±4 wt.-%, 13±4 wt.-% or 15±4 wt.-%, and in particular 11±2 wt.-%, 13±2 wt.-% or 15±2 wt.-%, based on the total weight of the pharmaceutical dosage form. In a further preferred embodiment, the content of nonionic surfactant is within the range of from 20±6 wt.-%, more preferably 20±5 wt.-%, still more preferably 20±4 wt.-%, most preferably 20±3 wt.-%, and in particular 20±2 wt.-%, based on the total weight of the pharmaceutical dosage form.

Preferably, the total amount of the nonionic surfactant that is contained in the pharmaceutical dosage form is within the range of from 0.01 to 200 mg, more preferably 0.1 to 190 mg, still more preferably 1.0 to 180 mg, yet more preferably 1.5 to 160 mg, most preferably 2.0 to 140 mg and in particular 2.5 to 120 mg.

In a preferred embodiment, the nonionic surfactant is contained in the pharmaceutical dosage form in an amount of 50±45 mg, 50±40 mg, 50±35 mg, 50±30 mg, 50±25 mg, 50±20 mg, 50±15 mg, 50±10 mg, or 50±5 mg. In another preferred embodiment, the nonionic surfactant is contained in the pharmaceutical dosage form in an amount of 70±65 mg, 70±60 mg, 70±55 mg, 70±50 mg, 70±45 mg, 70±40 mg, 70±35 mg, 70±30 mg, 70±25 mg, 70±20 mg, 70±15 mg, 70±10 mg, or 70±5 mg. In still another preferred embodiment, the nonionic surfactant is contained in the pharmaceutical dosage form in an amount of 90±85 mg, 90±80 mg, 90±75 mg, 90±70 mg, 90±65 mg, 90±60 mg, 90±55 mg, 90±50 mg, 90±45 mg, 90±40 mg, 90±35 mg, 90±30 mg, 90±25 mg, 90±20 mg, 90±15 mg, 90±10 mg, or 90±5 mg. In yet another preferred embodiment, the nonionic surfactant is contained in the pharmaceutical dosage form in an amount of 120±105 mg, 120±100 mg, 120±95 mg, 120±90 mg, 120±85 mg, 120±80 mg, 120±75 mg, 120±70 mg, 120±65 mg, 120±60 mg, 120±55 mg, 120±50 mg, 120±45 mg, 120±40 mg, 120±35 mg, 120±30 mg, 120±25 mg, 120±20 mg, 120±15 mg, 120±10 mg, or 120±5 mg.

In a preferred embodiment, the nonionic surfactant is contained in the pharmaceutical dosage form in an amount of 1.0±0.5 mg, 2.0±1.0 mg, 3.0±1.0 mg, 4.0±1.0 mg, 5.0±1.0 mg, 7.5±5 mg, 10±5 mg, 20±5 mg, 30±5 mg, 40±5 mg, 50±5 mg, 60±5 mg, 70±5 mg, 80±5 mg, 90±5 mg, 100±5 mg, 110±5 mg, 120±5 mg, 130±5 mg, 140±5 mg, 150±5 mg, or 160±5 mg. In still another preferred embodiment, the nonionic surfactant is contained in the pharmaceutical dosage form in an amount of 5±2.5 mg, 7.5±2.5 mg, 10±2.5 mg, 15±2.5 mg, 20±2.5 mg, 25±2.5 mg, 30±2.5 mg, 35±2.5 mg, 40±2.5 mg, 45±2.5 mg, 50±2.5 mg, 55±2.5 mg, 60±2.5 mg, 65±2.5 mg, 70±2.5 mg, 75±2.5 mg, 80±2.5 mg, 85±2.5 mg, 90±2.5 mg, 95±2.5 mg, 100±2.5 mg, 105±2.5 mg, 110±2.5 mg, 115±2.5 mg, 120±2.5 mg, 125±2.5 mg, 130±2.5 mg, 135±2.5 mg, 140±2.5 mg, 145±2.5 mg, 150±2.5 mg, 155±2.5 mg, or 160±2.5 mg.

Preferably, the relative weight ratio of the pharmacologically active compound and the nonionic surfactant is within the range of from 20:1 to 1:20, more preferably 15:1 to 1:15, still more preferably 10:1 to 1:10, yet more preferably 8:1 to 1:8, even more preferably 5:1 to 1:5, most preferably 3:1 to 1:3, and in particular 2:1 to 1:2.

The purpose of the nonionic surfactant that is contained in the pharmaceutical dosage form according to the invention is associated with the tamper resistance of the pharmaceutical dosage form, especially when the pharmaceutical dosage form is intended by an abuser for administration by a non-prescribed route, particularly intravenous administration of a liquid extract.

In a preferred embodiment, when
(i) subjecting a pharmaceutical dosage form (a) for 5 minutes in 5 mL of cold water, or (b) to boiling water and boiling the tablet for 5 minutes, respectively,
(ii) closing the vessel with aluminum foil, boiling extraction only,
(iii) drawing up the liquid into a syringe using a canula, preferably 0.80×40 mm BL/LB; 21 G×1½″, through a cigarette filter, and
(iv) determining the pharmacologically active compound content in the drawn liquid by HPLC analysis;
   the content of extracted pharmacologically active compound in the overhead liquid amounts to at most 14.5 wt.-%, 14.0 wt.-%, 13.5 wt.-%, or 13.0 wt.-%, more preferably at most 12.5 wt.-%, 12.0 wt.-%, 11.5 wt.-%, or 11.0 wt.-%, still more preferably at most 10.5 wt.-%, 10 wt.-%, 9.5 wt.-%, or 9.0 wt.-%, yet more preferably at most 8.5 wt.-%, 8.0 wt.-%, 7.5 wt.-%, or 7.0 wt.-%, even more preferably at most 6.5 wt.-%, 6.0 wt.-%, 5.5 wt.-%, or 5.0 wt.-%, most more preferably at most 4.5 wt.-%, 4.0 wt.-%, 3.5 wt.-%, or 3.0 wt.-%, and in particular at most 2.5 wt.-%, 2.0 wt.-%, 1.5 wt.-%, or 1.0 wt.-%, relative to the original total content of the pharmacologically active compound in the pharmaceutical dosage form, i.e. before it was subjected to the extraction test.

In a preferred embodiment, when
(i) subjecting a pharmaceutical dosage form (a) for 5 minutes in 5 mL of cold water, or (b) to boiling water and boiling the pharmaceutical dosage form for 5 minutes, respectively,
(ii) closing the vessel with aluminum foil, boiling extraction only,
(iii) drawing up the liquid into a syringe using a canula, preferably 0.80×40 mm BL/LB; 21 G×1½″, through a cigarette filter, and
(iv) determining the pharmacologically active compound content in the drawn liquid by HPLC analysis.
   the total amount of extracted pharmacologically active compound in the overhead liquid amounts to
at most 115 mg, 110 mg, 105 mg, or 100 mg, more preferably at most 95 mg, 90 mg, 85 mg, or 80 mg, still more preferably at most 75 mg, 70 mg, 65 mg, or 60 mg, yet more preferably at most 55 mg, 50 mg, 47.5 mg, or 45 mg, even more preferably at most 42.5 mg, 40 mg, 37.5 mg, or 35 mg, most more preferably at most 32.5 mg, 30 mg, 27.5 mg, or 25 mg, and in particular at most 22.5 mg, 20 mg, 17.5 mg, or 15 mg; or
at most 14.5 mg, 14.0 mg, 13.5 mg, or 13.0 mg, more preferably at most 12.5 mg, 12.0 mg, 11.5 mg, or 11.0 mg, still more preferably at most 10.5 mg, 10 mg, 9.5 mg, or 9.0 mg, yet more preferably at most 8.5 mg, 8.0 mg, 7.5 mg, or 7.0 mg, even more preferably at most 6.5 mg, 6.0 mg, 5.5 mg, or 5.0 mg, most more preferably at most 4.5 mg, 4.0 mg, 3.5 mg, or 3.0 mg, and in particular at most 2.5 mg, 2.0 mg, 1.5 mg, or 1.0 mg.

In a preferred embodiment, when
(i) subjecting a pharmaceutical dosage form (a) for 30 minutes to 30 mL of solvent with continuous shaking, or (b) giving a pharmaceutical dosage form in 30 mL of purified water, heating the water until boiling and shaking for 30 minutes, during the slow cooling of the water,
(ii) supplementing lost solvent, if any, and
(iii) determining the pharmacologically active compound content in the drawn liquid by HPLC analysis.
   the content of extracted pharmacologically active compound in the overhead liquid amounts to at most 30 wt.-%, preferably at most 27.5 wt.-% or at most 25 wt.-%, more preferably at most 25 wt.-% or at most 22.5 wt.-%, still more preferably at most 20 wt.-% or at most 17.5 wt.-%, yet more preferably at most 15 wt.-% or at most 14 wt.-%, most preferably more preferably at most 13 wt.-% or at most 12.5 wt.-%, and in particular at most 12.0 wt.-%, at most 11.0 wt.-%, or at most 10.0 wt.-%; and/or the total amount of extracted pharmacologically active compound in the overhead liquid amounts to at most 150 mg, 145 mg, 140 mg, or 135 mg, more preferably at most 130 mg, 125 mg, 120 mg, or 115 mg, still more preferably at most 110 mg, 105 mg, 100 mg, or 95 mg, yet more preferably at most 90 mg, 85 mg, 80 mg, or 75 mg, even more preferably at most 70 mg, 65 mg, 60 mg, or 55 mg, most more preferably at most 50 mg, 45 mg, 40 mg, or 37.5 mg, and in particular at most 35 mg, 32.5 mg, 30 mg, 27.5 mg or 25.0 mg.

Preferably, when a pharmaceutical dosage form according to the invention is treated with a commercial coffee mill, preferably type Bosch MKM6000, for 2 minutes, at least 50 wt.-%, more preferably at least 55 wt.-%, still more preferably at least 60 wt.-%, yet more preferably at least 65 wt.-%, even more preferably at least 70 wt.-%, most preferably at least 75 wt.-%, and in particular at least 80 wt.-%, of the total weight of the thus obtained material does not pass a sieve having a mesh size of 1.000 mm.

Preferably, when a pharmaceutical dosage form according to the invention is treated with a commercial coffee mill, preferably type Bosch MKM6000, for 2 minutes, it either remains intact and in one piece, or it is split into at most 10, preferably at most 7 or 8, more preferably at most 5 or 6, still more preferably at most 4, most preferably at most 3, and in particular at most 2 pieces.

In a preferred embodiment, the pharmaceutical dosage form according to the invention contains no substances which irritate the nasal passages and/or pharynx, i.e. substances which, when administered via the nasal passages and/or pharynx, bring about a physical reaction which is either so unpleasant for the patient that he/she does not wish to or cannot continue administration, for example burning, or physiologically counteracts taking of the corresponding active compound, for example due to increased nasal secretion or sneezing. Further examples of substances which irritate the nasal passages and/or pharynx are those which cause burning, itching, urge to sneeze, increased formation of secretions or a combination of at least two of these stimuli. Corresponding substances and the quantities thereof which are conventionally to be used are known to the person skilled in the art. Some of the substances which irritate the nasal passages and/or pharynx are accordingly based on one or more constituents or one or more plant parts of a hot substance drug. Corresponding hot substance drugs are known per se to the person skilled in the art and are described, for example, in “Pharmazeutische Biologie—Drogen and ihre Inhaltsstoffe” by Prof. Dr. Hildebert Wagner, 2nd., revised edition, Gustav Fischer Verlag, Stuttgart-New York, 1982, pages 82 et seq. The corresponding description is hereby introduced as a reference and is deemed to be part of the disclosure.

The pharmaceutical dosage form according to the invention furthermore preferably contains no emetic. Emetics are known to the person skilled in the art and may be present as such or in the form of corresponding derivatives, in particular esters or ethers, or in each case in the form of corresponding physiologically acceptable compounds, in particular in the form of the salts or solvates thereof. The pharmaceutical dosage form according to the invention preferably contains no emetic based on one or more constituents of ipecacuanha (ipecac) root, for example based on the constituent emetine, as are, for example, described in “Pharmazeutische Biologie—Drogen and ihre Inhaltsstoffe” by Prof. Dr. Hildebert Wagner, 2nd, revised edition, Gustav Fischer Verlag, Stuttgart, New York, 1982. The corresponding literature description is hereby introduced as a reference and is deemed to be part of the disclosure. The pharmaceutical dosage form according to the invention preferably also contains no apomorphine as an emetic.

The pharmaceutical dosage form according to the invention preferably also contains no bitter substance. Bitter substances and the quantities effective for use may be found in US-2003/0064099 A1, the corresponding disclosure of which should be deemed to be the disclosure of the present application and is hereby introduced as a reference. Examples of bitter substances are aromatic oils, such as peppermint oil, eucalyptus oil, bitter almond oil, menthol, fruit aroma substances, aroma substances from lemons, oranges, limes, grapefruit or mixtures thereof, and/or denatonium benzoate.

The pharmaceutical dosage form according to the invention accordingly preferably contains neither substances which irritate the nasal passages and/or pharynx, nor emetics, nor bitter substances.

Preferably, the pharmaceutical dosage form according to the invention contains no neuroleptics, for example a compound selected from the group consisting of haloperidol, promethacine, fluphenazine, perphenazine, levomepromazine, thioridazine, perazine, chlorpromazine, chlorprothixine, zuclopenthixol, flupentixol, prothipendyl, zotepine, benperidol, pipamperone, melperone and bromperidol.

In a preferred embodiment, the pharmaceutical dosage form according to the invention contains no pharmacologically active compound antagonists.

In another preferred embodiment, the pharmaceutical dosage form according to the invention does contain a pharmacologically active compound antagonist. Pharmacologically active compound antagonists suitable for a given pharmacologically active compound are known to the person skilled in the art and may be present as such or in the form of corresponding derivatives, in particular esters or ethers, or in each case in the form of corresponding physiologically acceptable compounds, in particular in the form of the salts or solvates thereof. The pharmaceutical dosage form according to the invention preferably contains an opioid as pharmacologically active compound and an opioid antagonist as pharmacologically active compound antagonist, wherein the opioid antagonist is selected from the group consisting of naloxone, naltrexone, nalmefene, nalide, nalmexone, nalorphine or naluphine, in each case optionally in the form of a corresponding physiologically acceptable compound, in particular in the form of a base, a salt or solvate. Naloxone and nalmexone as well as their physiologically acceptable salts are preferred pharmacologically active compound antagonists. The content of the pharmacologically active compound antagonist in the pharmaceutical dosage form is not limited.

Besides the pharmacologically active compound, the nonionic surfactant and the polyalkylene oxide the pharmaceutical dosage form according to the invention may contain further constituents, such as conventional pharmaceutical excipients.

Preferably, the pharmaceutical dosage form according to the invention contains a plasticizer.

The plasticizer improves the processability of the polyalkylene oxide. A preferred plasticizer is polyalkylene glycol, like polyethylene glycol, triacetin, fatty acids, fatty acid esters, waxes and/or microcrystalline waxes. Particularly preferred plasticizers are polyethylene glycols, such as PEG 6000.

Preferably, the content of the plasticizer is within the range of from 0.1 to 25 wt.-%, more preferably 0.5 to 22.5 wt.-%, still more preferably 1.0 to 20 wt.-%, yet more preferably 2.5 to 17.5 wt.-%, most preferably 5.0 to 15 wt.-% and in particular 7.5 to 12.5 wt.-%, based on the total weight of the pharmaceutical dosage form.

In a preferred embodiment, the plasticizer is a polyalkylene glycol having a content within the range of 5±4 wt.-%, more preferably 5±3.5 wt.-%, still more preferably 5±3 wt.-%, yet more preferably 5±2.5 wt.-%, most preferably 5±2 wt.-%, and in particular 5±1.5 wt.-%, based on the total weight of the pharmaceutical dosage form.

In another preferred embodiment, the plasticizer is a polyalkylene glycol having a content within the range of 10±8 wt.-%, more preferably 10±6 wt.-%, still more preferably 10±5 wt.-%, yet more preferably 10±4 wt.-%, most preferably 10±3 wt.-%, and in particular 10±2 wt.-%, based on the total weight of the pharmaceutical dosage form.

In still another preferred embodiment, the plasticizer is a polyalkylene glycol having a content within the range of 15±8 wt.-%, more preferably 15±6 wt.-%, still more preferably 15±5 wt.-%, yet more preferably 15±4 wt.-%, most preferably 15±3 wt.-%, and in particular 15±2 wt.-%, based on the total weight of the pharmaceutical dosage form.

The pharmaceutical dosage form according to the invention may further contain an antioxidant.

Suitable antioxidants include ascorbic acid, α-tocopherol (vitamin E), butylhydroxyanisol, butylhydroxytoluene, salts of ascorbic acid (vitamin C), ascorbylic palmitate, monothioglycerine, coniferyl benzoate, nordihydroguajaretic acid, gallus acid esters, phosphoric acid, and the derivatives thereof, such as vitamin E-succinate or vitamin E-palmitate and/or sodium bisulphite, more preferably butylhydroxytoluene (BHT) or butylhydroxyanisol (BHA) and/or α-tocopherol.

Preferably, the content of the antioxidant is within the range of from 0.001 to 5.0 wt.-%, more preferably 0.002 to 2.5 wt.-%, more preferably 0.003 to 1.5 wt.-%, still more preferably 0.005 to 1.0 wt.-%, yet more preferably 0.01 to 0.5 wt.-%, most preferably 0.05 to 0.4 wt.-% and in particular 0.1 to 0.3 wt.-%, based on the total weight of the pharmaceutical dosage form.

A particularly preferred antioxidant is α-tocopherol.

In a preferred embodiment, the content of α-tocopherol is within the range of 0.2±0.18 wt.-%, more preferably 0.2±0.15 wt.-%, still more preferably 0.2±0.12 wt.-%, yet more preferably 0.2±0.09 wt.-%, most preferably 0.2±0.06 wt.-%, and in particular 0.2±0.03 wt.-%, based on the total weight of the pharmaceutical dosage form.

In a preferred embodiment, when the pharmaceutical dosage form additionally comprises an acid, the relative weight ratio of the acid, preferably citric acid, and the antioxidant, preferably α-tocopherol, is within the range of from 10:1 to 1:10, more preferably 8:1 to 1:8, still more preferably 6:1 to 1:6, yet more preferably 5:1 to 1:4, most preferably 4:1 to 1:3 and in particular 3:1 to 1:2.

The pharmaceutical dosage form according to the invention may further contain a free physiologically acceptable acid in an amount of from 0.001 to 5.0 wt.-%, based on the total weight of the pharmaceutical dosage form. The acid may be organic or inorganic, liquid or solid. Solid acids are preferred, particularly crystalline organic or inorganic acids.

Preferably, the acid is free. This means that the acidic functional groups of the acid are not all together constituents of a salt of the pharmacologically active compound. If the pharmacologically active compound is present as a salt of an acid, e.g. as hydrochloride, the pharmaceutical dosage form according to the invention preferably contains as acid another, chemically different acid which is not present as a constituent of the salt of the pharmacologically active compound. In other words, monoacids that form a salt with the pharmacologically active compound are not to be considered as free acids in the meaning of the invention. When acid has more than a single acidic functional group (e.g. phosphoric acid), the acid may be present as a constituent of a salt of the pharmacologically active compound, provided that at least one of the acidic functional groups of the acid is not involved in the formation of the salt, i.e. is free. Preferably, however, each and every acidic functional group of acid is not involved in the formation of a salt with pharmacologically active compound. It is also possible, however, that free acid and the acid forming a salt with pharmacologically active compound are identical. Under these circumstances the acid is preferably present in molar excess compared to pharmacologically active compound.

In a preferred embodiment, the acid contains at least one acidic functional group (e.g. —CO₂H, —SO₃H, —PO₃H₂, —OH and the like) having a pK_{A} value within the range of 2.00±1.50, more preferably 2.00±1.25, still more preferably 2.00±1.00, yet more preferably 2.00±0.75, most preferably 2.00±0.50 and in particular 2.00±0.25. In another preferred embodiment, the acid contains at least one acidic functional group having a pK_{A} value within the range of 2.25±1.50, more preferably 2.25±1.25, still more preferably 2.25±1.00, yet more preferably 2.25±0.75, most preferably 2.25±0.50 and in particular 2.25±0.25. In another preferred embodiment, the acid contains at least one acidic functional group having a pK_{A} value within the range of 2.50±1.50, more preferably 2.50±1.25, still more preferably 2.50±1.00, yet more preferably 2.50±0.75, most preferably 2.50±0.50 and in particular 2.50±0.25. In another preferred embodiment, the acid contains at least one acidic functional group having a pK_{A} value within the range of 2.75±1.50, more preferably 2.75±1.25, still more preferably 2.75±1.00, yet more preferably 2.75±0.75, most preferably 2.75±0.50 and in particular 2.75±0.25. In another preferred embodiment, the acid contains at least one acidic functional group having a pK_{A} value within the range of 3.00±1.50, more preferably 3.00±1.25, still more preferably 3.00±1.00, yet more preferably 3.00±0.75, most preferably 3.00±0.50 and in particular 3.00±0.25. In still another preferred embodiment, the acid contains at least one acidic functional group having a pK_{A} value within the range of 3.25±1.50, more preferably 3.25±1.25, still more preferably 3.25±1.00, yet more preferably 3.25±0.75, most preferably 3.25±0.50 and in particular 3.25±0.25.

In yet another preferred embodiment, the acid contains at least one acidic functional group having a pK_{A} value within the range of 4.50±1.50, more preferably 4.50±1.25, still more preferably 4.50±1.00, yet more preferably 4.50±0.75, most preferably 4.50±0.50 and in particular 4.50±0.25. In yet another preferred embodiment, the acid contains at least one acidic functional group having a pK_{A} value within the range of 4.75±1.50, more preferably 4.75±1.25, still more preferably 4.75±1.00, yet more preferably 4.75±0.75, most preferably 4.75±0.50 and in particular 4.75±0.25. In yet another preferred embodiment, the acid contains at least one acidic functional group having a pK_{A} value within the range of 5.00±1.50, more preferably 5.00±1.25, still more preferably 5.00±1.00, yet more preferably 5.00±0.75, most preferably 5.00±0.50 and in particular 5.00±0.25.

Preferably, the acid is an organic carboxylic or sulfonic acid, particularly a carboxylic acid. Multicarboxylic acids and/or hydroxy-carboxylic acids are especially preferred.

In case of multicarboxylic acids, the partial salts thereof are also to be regarded as multi-carboxylic acids, e.g. the partial sodium, potassium or ammonium salts. For example, citric acid is a multicarboxylic acid having three carboxyl groups. As long as there remains at least one carboxyl group protonated (e.g. sodium dihydrogen citrate or disodium hydrogen citrate), the salt is to be regarded as a multicarboxylic acid. Preferably, however, all carboxyl groups of the multicarboxylic acid are protonated.

Preferably, the acid is of low molecular weight, i.e., not polymerized. Typically, the molecular weight of the acid is below 500 g/mol.

Examples of acids include saturated and unsaturated monocarboxylic acids, saturated and unsaturated bicarboxylic acids, tricarboxylic acids, α-hydroxyacids and β-hydroxylacids of monocarboxylic acids, α-hydroxyacids and β-hydroxyacids of bicarboxylic acids, α-hydroxy-acids and β-hydroxyacids of tricarboxylic acids, ketoacids, α-ketoacids, β-ketoacids, of the polycarboxylic acids, of the polyhydroxy monocarboxylic acids, of the polyhydroxy bicarboxylic acids, of the polyhydroxy tricarboxylic acids.

Preferably, the acid is selected from the group consisting of benzenesulfonic acid, citric acid, α-glucoheptonic acid, D-gluconic acid, glycolic acid, lactic acid, malic acid, malonic acid, mandelic acid, propanoic acid, succinic acid, tartaric acid (d, l, or dl), tosic acid (toluene-sulfonic acid), valeric acid, palmitic acid, pamoic acid, sebacic acid, stearic acid, lauric acid, acetic acid, adipic acid, glutaric acid, 4-chlorobenzenesulfonic acid, ethanedisulfonic acid, ethylsuccinic acid, fumaric acid, galactaric acid (mucic acid), D-glucuronic acid, 2-oxo-glutaric acid, glycerophosphoric acid, hippuric acid, isethionic acid (ethanolsulfonic acid), lactobionic acid, maleic acid, maleinic acid, 1,5-naphthalene-disulfonic acid, 2-naphthalene-sulfonic acid, pivalic acid, terephthalic acid, thiocyanic acid, cholic acid, n-dodecyl sulfate, 3-hydroxy-2-naphthoic acid, 1-hydroxy-2-naphthoic acid, oleic acid, undecylenic acid, ascorbic acid, (+)-camphoric acid, d-camphorsulfonic acid, dichloroacetic acid, ethanesulfonic acid, formic acid, methanesulfonic acid, nicotinic acid, orotic acid, oxalic acid, picric acid, L-pyroglutamic acid, saccharine, salicylic acid, gentisic acid, and/or 4-acetamidobenzoic acid.

The content of the acid is preferably within the range of from 0.001 to 5.0 wt.-%, preferably 0.005 to 2.5 wt.-%, more preferably 0.01 to 2.0 wt.-%, still more preferably 0.05 to 1.5 wt.-%, most preferably 0.1 to 1.0 wt.-% and in particular 0.2 to 0.9 wt.-%, based on the total weight of the pharmaceutical dosage form.

Preferably, the acid is a multicarboxylic acid. More preferably, the multicarboxylic acid is selected from the group consisting of citric acid, maleic acid and fumaric acid.

Citric acid is particularly preferred.

The multicarboxylic acid, preferably citric acid, may be present in its anhydrous form or as a solvate and hydrate, respectively, e.g., as monohydrate.

In a preferred embodiment, the content of the acid, preferably citric acid, is within the range of 0.2±0.18 wt.-%, more preferably 0.2±0.15 wt.-%, still more preferably 0.2±0.12 wt.-%, yet more preferably 0.2±0.09 wt.-%, most preferably 0.2±0.06 wt.-%, and in particular 0.2±0.03 wt.-%, based on the total weight of the pharmaceutical dosage form.

In another preferred embodiment, the content of the acid, preferably citric acid, is within the range of 0.3±0.18 wt.-%, more preferably 0.3±0.15 wt.-%, still more preferably 0.3±0.12 wt.-%, yet more preferably 0.3±0.09 wt.-%, most preferably 0.3±0.06 wt.-%, and in particular 0.3±0.03 wt.-%, based on the total weight of the pharmaceutical dosage form.

In still another preferred embodiment, the content of the acid, preferably citric acid, is within the range of 0.4±0.18 wt.-%, more preferably 0.4±0.15 wt.-%, still more preferably 0.4±0.12 wt.-%, yet more preferably 0.4±0.09 wt.-%, most preferably 0.4±0.06 wt.-%, and in particular 0.4±0.03 wt.-%, based on the total weight of the pharmaceutical dosage form.

In yet another preferred embodiment, the content of the acid, preferably citric acid, is within the range of 0.5±0.18 wt.-%, more preferably 0.5±0.15 wt.-%, still more preferably 0.5±0.12 wt.-%, yet more preferably 0.5±0.09 wt.-%, most preferably 0.5±0.06 wt.-%, and in particular 0.5±0.03 wt.-%, based on the total weight of the pharmaceutical dosage form.

In yet another preferred embodiment, the content of the acid, preferably citric acid, is within the range of 0.6±0.18 wt.-%, more preferably 0.6±0.15 wt.-%, still more preferably 0.6±0.12 wt.-%, yet more preferably 0.6±0.09 wt.-%, most preferably 0.6±0.06 wt.-%, and in particular 0.6±0.03 wt.-%, based on the total weight of the pharmaceutical dosage form.

In yet another preferred embodiment, the content of the acid, preferably citric acid, is within the range of 0.7±0.18 wt.-%, more preferably 0.7±0.15 wt.-%, still more preferably 0.7±0.12 wt.-%, yet more preferably 0.7±0.09 wt.-%, most preferably 0.7±0.06 wt.-%, and in particular 0.7±0.03 wt.-%, based on the total weight of the pharmaceutical dosage form.

In yet another preferred embodiment, the content of acid, preferably citric acid, is within the range of 0.8±0.18 wt.-%, more preferably 0.8±0.15 wt.-%, still more preferably 0.8±0.12 wt.-%, yet more preferably 0.8±0.09 wt.-%, most preferably 0.8±0.06 wt.-%, and in particular 0.8±0.03 wt.-%, based on the total weight of the pharmaceutical dosage form.

In yet another preferred embodiment, the content of the acid, preferably citric acid, is within the range of 0.85±0.18 wt.-%, more preferably 0.85±0.15 wt.-%, still more preferably 0.85±0.12 wt.-%, yet more preferably 0.85±0.09 wt.-%, most preferably 0.85±0.06 wt.-%, and in particular 0.85±0.03 wt.-%, based on the total weight of the pharmaceutical dosage form.

In still another preferred embodiment, the content of the acid, preferably citric acid, is within the range of 0.9±0.18 wt.-%, more preferably 0.9±0.15 wt.-%, still more preferably 0.9±0.12 wt.-%, yet more preferably 0.9±0.09 wt.-%, most preferably 0.9±0.06 wt.-%, and in particular 0.9±0.03 wt.-%, based on the total weight of the pharmaceutical dosage form.

In a further preferred embodiment, the content of the acid, preferably citric acid, is within the range of 1.0±0.18 wt.-%, more preferably 1.0±0.15 wt.-%, still more preferably 1.0±0.12 wt.-%, yet more preferably 1.0±0.09 wt.-%, most preferably 1.0±0.06 wt.-%, and in particular 1.0±0.03 wt.-%, based on the total weight of the pharmaceutical dosage form.

The pharmaceutical dosage form according to the invention may also contain a natural, semi-synthetic or synthetic wax. Waxes with a softening point of at least 50° C., more preferably 60° C. are preferred. Carnauba wax and beeswax are particularly preferred, especially carnauba wax.

Preferably, the pharmaceutical dosage form according to the invention contains a coating, preferably a film-coating. Suitable coating materials are known to the skilled person. Suitable coating materials are commercially available, e.g. under the trademarks Opadry® and Eudragit®.

Examples of suitable materials include cellulose esters and cellulose ethers, such as methylcellulose (MC), hydroxypropylmethylcellulose (HPMC), hydroxypropylcellulose (HPC), hydroxyethylcellulose (HEC), sodium carboxymethylcellulose (Na—CMC), ethylcellulose (EC), cellulose acetate phthalate (CAP), hydroxypropylmethylcellulose phthalate (HPMCP); poly(meth)acrylates, such as aminoalkylmethacrylate copolymers, ethylacrylate methylmethacrylate copolymers, methacrylic acid methylmethacrylate copolymers, methacrylic acid methylmethacrylate copolymers; vinyl polymers, such as polyvinylpyrrolidone, polyvinylacetatephthalate, polyvinyl alcohol, polyvinylacetate; and natural film formers, such as shellack.

In a particularly preferred embodiment, the coating is water-soluble. In a preferred embodiment, the coating is based on polyvinyl alcohol, such as polyvinyl alcohol-part, hydrolyzed, and may additionally contain polyethylene glycol, such as macrogol 3350, and/or pigments. In another preferred embodiment, the coating is based on hydroxypropylmethyl-cellulose, preferably hypromellose type 2910 having a viscosity of 3 to 15 mPas.

The coating of the pharmaceutical dosage form can increase its storage stability.

The coating can be resistant to gastric juices and dissolve as a function of the pH value of the release environment. By means of this coating, it is possible to ensure that the pharmaceutical dosage form according to the invention passes through the stomach undissolved and the active compound is only released in the intestines. The coating which is resistant to gastric juices preferably dissolves at a pH value of between 5 and 7.5. Corresponding materials and methods for the delayed release of active compounds and for the application of coatings which are resistant to gastric juices are known to the person skilled in the art, for example from “Coated Pharmaceutical dosage forms—Fundamentals, Manufacturing Techniques, Biopharmaceutical Aspects, Test Methods and Raw Materials” by Kurt H. Bauer, K. Lehmann, Hermann P. Osterwald, Rothgang, Gerhart, 1st edition, 1998, Medpharm Scientific Publishers.

The pharmaceutical dosage form according to the invention is preferably tamper-resistant. Preferably, tamper-resistance is achieved based on the mechanical properties of the pharmaceutical dosage form so that comminution is avoided or at least substantially impeded. According to the invention, the term comminution means the pulverization of the pharmaceutical dosage form using conventional means usually available to an abuser, for example a pestle and mortar, a hammer, a mallet or other conventional means for pulverizing under the action of force. Thus, tamper-resistance preferably means that pulverization of the pharmaceutical dosage form using conventional means is avoided or at least substantially impeded.

Preferably, the mechanical properties of the pharmaceutical dosage form according to the invention, particularly its breaking strength, substantially rely on the presence and spatial distribution of the polyalkylene oxide, although its mere presence does typically not suffice in order to achieve said properties. The advantageous mechanical properties of the pharmaceutical dosage form according to the invention may not automatically be achieved by simply processing pharmacologically active compound, nonionic surfactant, polyalkylene oxide, and optionally further excipients by means of conventional methods for the preparation of pharmaceutical dosage forms. In fact, usually suitable apparatuses must be selected for the preparation and critical processing parameters must be adjusted, particularly pressure/force, temperature and time. Thus, even if conventional apparatuses are used, the process protocols usually must be adapted in order to meet the required criteria.

Furthermore, tamper-resistance is achieved based on the poor solubility properties of the pharmaceutical dosage form in alcohol, especially ethanol, thereby effectively preventing alcohol dose dumping.

The pharmaceutical dosage form according to the invention has a breaking strength of at least 500 N, preferably at least 750 N, more preferably at least 1000 N, most preferably at least 1250 N and in particular at least 1500 N.

The “breaking strength” (resistance to crushing) of a pharmaceutical dosage form is known to the skilled person. In this regard it can be referred to, e.g., W. A. Ritschel, Die Tablette, 2. Auflage, Editio Cantor Verlag Aulendorf, 2002; H Liebermann et al., Pharmaceutical dosage forms: Tablets, Vol. 2, Informa Healthcare; 2 edition, 1990; and Encyclopedia of Pharmaceutical Technology, Informa Healthcare; 1 edition.

For the purpose of the specification, the breaking strength is preferably defined as the amount of force that is necessary in order to fracture the pharmaceutical dosage form (=breaking force). Therefore, for the purpose of the specification the pharmaceutical dosage form does preferably not exhibit the desired breaking strength when it breaks, i.e., is fractured into at least two independent parts that are separated from one another. In another preferred embodiment, however, the pharmaceutical dosage form is regarded as being broken if the force decreases by 25% (threshold value) of the highest force measured during the measurement (see below).

The pharmaceutical dosage forms according to the invention are distinguished from conventional pharmaceutical dosage forms in that, due to their breaking strength, they cannot be pulverized by the application of force with conventional means, such as for example a pestle and mortar, a hammer, a mallet or other usual means for pulverization, in particular devices developed for this purpose (tablet crushers). In this regard “pulverization” means crumbling into small particles that would immediately release the pharmacologically active compound in a suitable medium. Avoidance of pulverization virtually rules out oral or parenteral, in particular intravenous or nasal abuse.

Conventional tablets typically have a breaking strength well below 200 N in any direction of extension. The breaking strength of conventional round tablets may be estimated according to the following empirical formula: Breaking Strength [in N]=10× Diameter Of The Tablet [in mm]. Thus, according to said empirical formula, a round tablet having a breaking strength of at least 300 N would require a diameter of at least 30 mm). Such a tablet, however, could not be swallowed. The above empirical formula preferably does not apply to the pharmaceutical dosage forms of the invention, which are not conventional but rather special.

Further, the actual mean chewing force is about 220 N (cf., e.g., P. A. Proeschel et al., J Dent Res, 2002, 81(7), 464-468). This means that conventional tablets having a breaking strength well below 200 N may be crushed upon spontaneous chewing, whereas the pharmaceutical dosage forms according to the invention may not.

Still further, when applying a gravitational acceleration of about 9.81 m/s², 300 N correspond to a gravitational force of more than 30 kg, i.e. the pharmaceutical dosage forms according to the invention can preferably withstand a weight of more than 30 kg without being pulverised.

Methods for measuring the breaking strength of a pharmaceutical dosage form are known to the skilled artisan. Suitable devices are commercially available.

For example, the breaking strength (resistance to crushing) can be measured in accordance with the Eur. Ph. 5.0, 2.9.8 or 6.0, 2.09.08 “Resistance to Crushing of Tablets”. The test is intended to determine, under defined conditions, the resistance to crushing of tablets, measured by the force needed to disrupt them by crushing. The apparatus consists of 2 jaws facing each other, one of which moves towards the other. The flat surfaces of the jaws are perpendicular to the direction of movement. The crushing surfaces of the jaws are flat and larger than the zone of contact with the tablet. The apparatus is calibrated using a system with a precision of 1 Newton. The tablet is placed between the jaws, taking into account, where applicable, the shape, the break-mark and the inscription; for each measurement the tablet is oriented in the same way with respect to the direction of application of the force (and the direction of extension in which the breaking strength is to be measured). The measurement is carried out on 10 tablets, taking care that all fragments of tablets have been removed before each determination. The result is expressed as the mean, minimum and maximum values of the forces measured, all expressed in Newton.

A similar description of the breaking strength (breaking force) can be found in the USP. The breaking strength can alternatively be measured in accordance with the method described therein where it is stated that the breaking strength is the force required to cause a tablet to fail (i.e., break) in a specific plane. The tablets are generally placed between two platens, one of which moves to apply sufficient force to the tablet to cause fracture. For conventional, round (circular cross-section) tablets, loading occurs across their diameter (sometimes referred to as diametral loading), and fracture occurs in the plane. The breaking force of tablets is commonly called hardness in the pharmaceutical literature; however, the use of this term is misleading. In material science, the term hardness refers to the resistance of a surface to penetration or indentation by a small probe. The term crushing strength is also frequently used to describe the resistance of tablets to the application of a compressive load. Although this term describes the true nature of the test more accurately than does hardness, it implies that tablets are actually crushed during the test, which is often not the case.

Alternatively, the breaking strength (resistance to crushing) can be measured in accordance with WO 2005/016313, WO 2005/016314, and WO 2006/082099, which can be regarded as a modification of the method described in the Eur. Ph. The apparatus used for the measurement is preferably a “Zwick Z 2.5” materials tester, Fₘₐₓ=2.5 kN with a maximum draw of 1150 mm, which should be set up with one column and one spindle, a clearance behind of 100 mm and a test speed adjustable between 0.1 and 800 mm/min together with testControl software. Measurement is performed using a pressure piston with screw-in inserts and a cylinder (diameter 10 mm), a force transducer, Fₘₐₓ. 1 kN, diameter=8 mm, class 0.5 from 10 N, class 1 from 2 N to ISO 7500-1, with manufacturers test certificate M according to DIN 55350-18 (Zwick gross force Fₘₐₓ=1.45 kN) (all apparatus from Zwick GmbH & Co. KG, Ulm, Germany) with Order No BTC-FR 2.5 TH. D09 for the tester, Order No BTC-LC 0050N. P01 for the force transducer, Order No BO 70000 S06 for the centring device.

In a preferred embodiment of the invention, the breaking strength is measured by means of a breaking strength tester e.g. Sotax®, type HT100 or type HT1 (Allschwil, Switzerland). Both, the Sotax® HT100 and the Sotax® HT1 can measure the breaking strength according to two different measurement principles: constant speed (where the test jaw is moved at a constant speed adjustable from 5-200 mm/min) or constant force (where the test jaw increases force linearly adjustable from 5-100 N/sec). In principle, both measurement principles are suitable for measuring the breaking strength of the pharmaceutical dosage form according to the invention. Preferably, the breaking strength is measured at constant speed, preferably at a constant speed of 120 mm/min.

In a preferred embodiment, the pharmaceutical dosage form is regarded as being broken if it is fractured into at least two separate pieces.

The pharmaceutical dosage form according to the invention preferably exhibits mechanical strength over a wide temperature range, in addition to the breaking strength (resistance to crushing) optionally also sufficient hardness, impact resistance, impact elasticity, tensile strength and/or modulus of elasticity, optionally also at low temperatures (e.g. below −24° C., below −40° C. or in liquid nitrogen), for it to be virtually impossible to pulverize by spontaneous chewing, grinding in a mortar, pounding, etc. Thus, preferably, the comparatively high breaking strength of the pharmaceutical dosage form according to the invention is maintained even at low or very low temperatures, e.g., when the pharmaceutical dosage form is initially chilled to increase its brittleness, for example to temperatures below −25° C., below −40° C. or even in liquid nitrogen.

The pharmaceutical dosage form according to the invention is characterized by a certain degree of breaking strength. This does not mean that the pharmaceutical dosage form must also exhibit a certain degree of hardness. Hardness and breaking strength are different physical properties. Therefore, the tamper resistance of the pharmaceutical dosage form does not necessarily depend on the hardness of the pharmaceutical dosage form. For instance, due to its breaking strength, impact strength, elasticity modulus and tensile strength, respectively, the pharmaceutical dosage form can preferably be deformed, e.g. plastically, when exerting an external force, for example using a hammer, but cannot be pulverized, i.e., crumbled into a high number of fragments. In other words, the pharmaceutical dosage form according to the invention is characterized by a certain degree of breaking strength, but not necessarily also by a certain degree of form stability.

Therefore, in the meaning of the specification, a pharmaceutical dosage form that is deformed when being exposed to a force in a particular direction of extension but that does not break (plastic deformation or plastic flow) is preferably to be regarded as having the desired breaking strength in said direction of extension.

Preferably, the pharmaceutical dosage form for oral administration
has a breaking strength of at least 500 N, more preferably at least 750 N, yet more preferably at least 1000 N, most preferably at least 1500 N; and/or
comprises a pharmacologically active compound selected from opioids, more preferably from hydromorphone, oxycodone, oxymorphone, tapentadol and the physiologically acceptable salts thereof; and/or
comprises a nonionic surfactant according to general formula (I-a) and/or a nonionic surfactant according to general formula (I-b); and/or
is configured for oral administration twice daily; and/or
contains at least 30 wt.-%, more preferably at least 35 wt.-%, still more preferably at least 40 wt.-% of a polyalkylene oxide having an average molecular weight of at least 500,000 g/mol, more preferably at least 1,000,000 g/mol, relative to the total weight of the pharmaceutical dosage form; and/or
optionally, contains a plasticizer, preferably polyethylene glycol; and/or
optionally, contains an antioxidant, preferably a-tocopherol; and/or
optionally, contains a free acid, preferably citric acid; and/or
optionally, contains an additional matrix polymer, preferably a cellulose ether, more preferably HPMC; and/or
optionally contains a further polymer obtainable by polymerization of a monomer composition comprising an ethylenically unsaturated monomer bearing an anionic functional group, in protonated form or a physiologically acceptable salt thereof.

The pharmaceutical dosage form according to the invention may be produced by different processes, the particularly preferred of which are explained in greater detail below. Several suitable processes have already been described in the prior art. In this regard it can be referred to, e.g., WO 2005/016313, WO 2005/016314, WO 2005/063214, WO 2005/102286, WO 2006/002883, WO 2006/002884, WO 2006/002886, WO 2006/082097, and WO 2006/082099.

The invention also relates to pharmaceutical dosage forms that are obtainable by any of the processes described here below.

In general, the process for the production of the pharmaceutical dosage form according to the invention preferably comprises the following steps:
(a) mixing all ingredients;
(b) optionally pre-forming the mixture obtained from step (a), preferably by applying heat and/or force to the mixture obtained from step (a), the quantity of heat supplied preferably not being sufficient to heat the polyalkylene oxide up to its softening point;
(c) hardening the mixture by applying heat and force, it being possible to supply the heat during and/or before the application of force and the quantity of heat supplied being sufficient to heat the polyalkylene oxide at least up to its softening point;
(d) optionally singulating the hardened mixture;
(e) optionally shaping the pharmaceutical dosage form; and
(f) optionally providing a film coating.

Heat may be supplied directly, e.g. by contact or by means of hot gas such as hot air, or with the assistance of ultrasound, microwaves and/or radiation. Force may be applied and/or the pharmaceutical dosage form may be shaped for example by direct tabletting or with the assistance of a suitable extruder, particularly by means of a screw extruder equipped with two screws (twin-screw-extruder) or by means of a planetary gear extruder.

The final shape of the pharmaceutical dosage form may either be provided during the hardening of the mixture by applying heat and force (step (c)) or in a subsequent step (step (e)). In both cases, the mixture of all components is preferably in the plastified state, i.e. preferably, shaping is performed at a temperature at least above the softening point of the polyalkylene oxide. However, extrusion at lower temperatures, e.g. ambient temperature, is also possible and may be preferred.

Shaping can be performed, e.g., by means of a tabletting press comprising die and punches of appropriate shape.

A particularly preferred process for the manufacture of the pharmaceutical dosage form of the invention involves hot-melt extrusion. In this process, the pharmaceutical dosage form according to the invention is produced by thermoforming with the assistance of an extruder, preferably without there being any observable consequent discoloration of the extrudate. It has been surprisingly found that acid is capable of suppressing discoloration. In the absence of acid, the extrudate tends to develop beige to yellowish coloring whereas in the presence of acid the extrudates are substantially colorless, i.e. white.

This process is characterized in that
a) all components are mixed,
b) the resultant mixture is heated in the extruder at least up to the softening point of the polyalkylene oxide and extruded through the outlet orifice of the extruder by application of force,
c) the still plastic extrudate is singulated and formed into the pharmaceutical dosage form or
d) the cooled and optionally reheated singulated extrudate is formed into the pharmaceutical dosage form.

Mixing of the components according to process step a) may also proceed in the extruder.

The components may also be mixed in a mixer known to the person skilled in the art. The mixer may, for example, be a roll mixer, shaking mixer, shear mixer or compulsory mixer.

Before blending with the remaining components, polyalkylene oxide (is preferably provided according to the invention with an antioxidant, preferably α-tocopherol. This may proceed by mixing the two components, the polyalkylene oxide and the antioxidant, preferably by dissolving or suspending the antioxidant in a highly volatile solvent and homogeneously mixing this solution or suspension with polyalkylene oxide and removing the solvent by drying, preferably under an inert gas atmosphere.

The preferably molten, mixture which has been heated in the extruder at least up to the softening point of polyalkylene oxide is extruded from the extruder through a die with at least one bore.

The process according to the invention requires the use of suitable extruders, preferably screw extruders. Screw extruders which are equipped with two screws (twin-screw-extruders) are particularly preferred.

The extrusion is preferably performed so that the expansion of the strand due to extrusion is not more than 30%, i.e. that when using a die with a bore having a diameter of e.g. 6 mm, the extruded strand should have a diameter of not more than 8 mm. More preferably, the expansion of the strand is not more than 25%, still more preferably not more than 20%, most preferably not more than 15% and in particular not more than 10%.

Preferably, extrusion is performed in the absence of water, i.e., no water is added. However, traces of water (e.g., caused by atmospheric humidity) may be present.

The extruder preferably comprises at least two temperature zones, with heating of the mixture at least up to the softening point of the polyalkylene oxide proceeding in the first zone, which is downstream from a feed zone and optionally mixing zone. The throughput of the mixture is preferably from 1.0 kg to 15 kg/hour. In a preferred embodiment, the throughput is from 1 to 3.5 kg/hour. In another preferred embodiment, the throughput is from 4 to 15 kg/hour.

In a preferred embodiment, the die head pressure is within the range of from 25 to 100 bar. In another preferred embodiment, the die head pressure is within the range of from 3 to 25 bar. The die head pressure can be adjusted inter alia by die geometry, temperature profile and extrusion speed.

The die geometry or the geometry of the bores is freely selectable. The die or the bores may accordingly exhibit a round, oblong or oval cross-section, wherein the round cross-section preferably has a diameter of 0.1 mm to 15 mm and the oblong cross-section preferably has a maximum lengthwise extension of 21 mm and a crosswise extension of 10 mm. Preferably, the die or the bores have a round cross-section. The casing of the extruder used according to the invention may be heated or cooled. The corresponding temperature control, i.e. heating or cooling, is so arranged that the mixture to be extruded exhibits at least an average temperature (product temperature) corresponding to the softening temperature of the polyalkylene oxide and does not rise above a temperature at which the pharmacologically active compound to be processed may be damaged. Preferably, the temperature of the mixture to be extruded is adjusted to below 180° C., preferably below 150° C., but at least to the softening temperature of polyalkylene oxide. Typical extrusion temperatures are 120° C. and 130° C.

In a preferred embodiment, the extruder torque is within the range of from 30 to 95%. Extruder torque can be adjusted inter alia by die geometry, temperature profile and extrusion speed.

After extrusion of the molten mixture and optional cooling of the extruded strand or extruded strands, the extrudates are preferably singulated. This singulation may preferably be performed by cutting up the extrudates by means of revolving or rotating knives, water jet cutters, wires, blades or with the assistance of laser cutters.

Preferably, intermediate or final storage of the optionally singulated extrudate or the final shape of the pharmaceutical dosage form according to the invention is performed under oxygen-free atmosphere which may be achieved, e.g., by means of oxygen-scavengers.

The singulated extrudate may be press-formed into tablets in order to impart the final shape to the pharmaceutical dosage form.

The application of force in the extruder onto the at least plasticized mixture is adjusted by controlling the rotational speed of the conveying device in the extruder and the geometry thereof and by dimensioning the outlet orifice in such a manner that the pressure necessary for extruding the plasticized mixture is built up in the extruder, preferably immediately prior to extrusion. The extrusion parameters which, for each particular composition, are necessary to give rise to a pharmaceutical dosage form with desired mechanical properties, may be established by simple preliminary testing.

For example but not limiting, extrusion may be performed by means of a twin-screw-extruder type ZSE 18 or ZSE27 (Leistritz, Nurnberg, Germany), screw diameters of 18 or 27 mm. Screws having eccentric ends may be used. A heatable die with a round bore having a diameter of 4, 5, 6, 7, 8, 9, or 10 mm may be used. The extrusion parameters may be adjusted e.g. to the following values: rotational speed of the screws: 120 Upm; delivery rate 2 kg/h for a ZSE 18 or 8 kg/h for a ZSE27; product temperature: in front of die 125° C.; temperature of the die 135° C.; and jacket temperature: 110° C.

Preferably, extrusion is performed by means of twin-screw-extruders or planetary-gear-extruders, twin-screw extruders (co-rotating or contra-rotating) being particularly preferred.

The pharmaceutical dosage form according to the invention is preferably produced by thermoforming with the assistance of an extruder without any observable consequent discoloration of the extrudates.

The process for the preparation of the pharmaceutical dosage form according to the invention is preferably performed continuously. Preferably, the process involves the extrusion of a homogeneous mixture of all components. It is particularly advantageous if the thus obtained intermediate, e.g. the strand obtained by extrusion, exhibits uniform properties. Particularly desirable are uniform density, uniform distribution of the active compound, uniform mechanical properties, uniform porosity, uniform appearance of the surface, etc. Only under these circumstances the uniformity of the pharmacological properties, such as the stability of the release profile, may be ensured and the amount of rejects can be kept low.

A further aspect of the invention relates to the use of a pharmacologically active compound in combination with a nonionic surfactant for the manufacture of the pharmaceutical dosage form as described above for the treatment of pain, preferably moderate to severe pain such as moderate to severe low back pain.

A further aspect of the invention relates to the use of a pharmaceutical dosage form as described above for avoiding or hindering the abuse of the pharmacologically active compound contained therein.

A further aspect of the invention relates to the use of a pharmaceutical dosage form as described above for avoiding or hindering the unintentional overdose of the pharmacologically active compound contained therein.

In this regard, the invention also relates to the use of a pharmacologically active compound as described above and/or a polyalkylene oxide as described above for the manufacture of the pharmaceutical dosage form according to the invention for the prophylaxis and/or the treatment of a disorder, thereby preventing an overdose of the pharmacologically active compound, particularly due to comminution of the pharmaceutical dosage form by mechanical action.

Further, the invention relates to a method for the prophylaxis and/or the treatment of a disorder comprising the administration of the pharmaceutical dosage form according to the invention, thereby preventing an overdose of the pharmacologically active compound, particularly due to comminution of the pharmaceutical dosage form by mechanical action. Preferably, the mechanical action is selected from the group consisting of chewing, grinding in a mortar, pounding, and using apparatuses for pulverizing conventional pharmaceutical dosage forms.

The following examples further illustrate the invention but are not to be construed as limiting its scope.

### General Procedure:

Polyethylene oxide, tramadol hydrochloride, and all other excipients were weighted and sieved to each other.

The powder was mixed and dosed gravimetrically to an extruder. Hot-melt extrusion (revolution speed 100 rpm) was performed by means of a twin screw extruder of type ZSE27 PH 40D Micro (Leistritz, Nürnberg, Germany) that was equipped with a heatable round die either having a diameter of 6 mm (cutting length 14.4 mm) or having a diameter of 10 mm (cutting length 6.6 mm).

The hot extrudate was cooled by ambient air and the cooled extrusion strand was comminuted to cut pieces. The cut pieces were shaped by means of an excenter press which was equipped with punches of various size and shape.

The breaking strength of the pharmaceutical dosage forms was measured by means of a Sotax® HT100. A tablet was regarded as failing the breaking strength test when during the measurement the force dropped below the threshold value of 25% of the maximum force that was observed during the measurement, regardless of whether the pharmaceutical dosage form was fractured into separate pieces or not. All values are given as a mean of 10 measurements.

The in vitro release profile of tramadol hydrochloride was measured in 600 ml phosphate buffer (pH 6.8) at temperature of 37° C. with sinker (type 1 or 2). The rotation speed of the paddle was adjusted to 75/min.

### EXAMPLE 1

### a) Composition

Tablets having the following compositions were prepared:

| | C-1 | | I-1 | | I-2 | |
|---|---|---|---|---|---|---|
| | Mg | wt.-% | mg | wt.-% | mg | wt.-% |
| Tramadol HCl | 80.0 | 13.3 | 80.0 | 13.3 | 80.0 | 13.3 |
| Polyethylene Oxide | 370.0 | 61.7 | 370.0 | 61.7 | 370.0 | 61.7 |
| M_{w} 7 × 10⁶ | | | | | | |
| HPMC 100,000 | 60.0 | 10.0 | 60.0 | 10.0 | 60.0 | 10.0 |
| mPa · s | | | | | | |
| Macrogol 6,000 | 90.0 | 15.0 | — | — | — | — |
| Poloxamer 407 | — | — | 90.0 | 15.0 | — | — |
| (Lutrol ® F127) | | | | | | |
| Poloxamer 188 | — | — | — | — | 90.0 | 15.0 |
| (Lutrol ® F68) | | | | | | |
| Σ | 600.0 | 100.0 | 600.0 | 100.0 | 600.0 | 100.0 |

### b) Hot-Melt Extrusion

The following extrusion parameters were adjusted and measured, respectively:

| | C-1′ | I-1′ | I-2′ | I-1″ | I-2″ |
|---|---|---|---|---|---|
| diameter of die [mm] | 10 | 10 | 10 | 6 | 6 |
| throughput [kg/h] | 3.5 | 3.5 | 3.5 | 3.5 | 3.5 |
| melt temperature [° C.] | 102 | 108 | 116 | 117 | 120 |
| performance (%) | 52 | 43 | 53 | 48 | 50 |
| melt pressure [bar] | 29 | 20 | 38 | 43 | 50 |
| strand diameter [mm] | 10.2 | 10.3 | 10.3 | 6.9 | 7.2 |
| cutting length [mm] | 6.7 | 6.7 | 6.5 | 14.5 | 14.3 |

Crude extrudates having the following weights and dimensions were obtained:

| n = 10 | | C-1′ | I-1′ | I-2′ | I-1″ | I-2″ |
|---|---|---|---|---|---|---|
| weight [mg] | min | 595 | 595 | 588 | 595 | 595 |
| | max | 610 | 616 | 599 | 608 | 603 |
| | average | 603 | 603 | 594 | 599 | 601 |
| length [mm] | min | 6.55 | 6.71 | 6.71 | 14.22 | 14.05 |
| | max | 6.81 | 6.89 | 6.83 | 14.42 | 14.20 |
| | average | 6.68 | 6.77 | 6.79 | 14.30 | 14.14 |
| diameter [mm] | min | 9.98 | 9.77 | 9.94 | 6.95 | 7.02 |
| | max | 10.17 | 10.42 | 10.45 | 7.09 | 7.20 |
| | average | 10.10 | 10.17 | 10.20 | 7.02 | 7.11 |

### c) Formation of Tablets from Extrudates

Tablets were manufactured from the crude extrudates by means of a round punch and an oblong punch, respectively, having the following dimensions (no engraving):

| Example | Form of punch |
|---|---|
| round | biconvex, round, diameter 12 mm, radius of curvature 9 mm |
| oblong | biconvex, oblong, 7.5 × 18.0 mm |

Tablets having the following weights and dimensions were obtained:

| n = 10 | | C-1′_{round} | I-1′_{round} | I-2′_{round} | I-1″_{oblong} | I-2″_{oblong} |
|---|---|---|---|---|---|---|
| length | min | — | — | — | 16.96 | 16.80 |
| [mm] | max | — | — | — | 17.14 | 17.17 |
| | average | — | — | — | 17.02 | 16.92 |
| width | min | — | — | — | 7.52 | 7.51 |
| [mm] | max | — | — | — | 7.56 | 7.54 |
| | average | — | — | — | 7.54 | 7.53 |
| thick- | min | 6.43 | 6.51 | 6.44 | 5.26 | 5.29 |
| ness | max | 6.58 | 6.60 | 6.82 | 5.38 | 5.48 |
| [mm] | average | 6.53 | 6.56 | 6.61 | 5.35 | 5.41 |
| dia- | min | 11.63 | 11.58 | 11.75 | — | — |
| meter | max | 11.79 | 11.87 | 11.82 | — | — |
| [mm] | average | 11.69 | 11.76 | 11.80 | — | — |

### d) In-Vitro Release

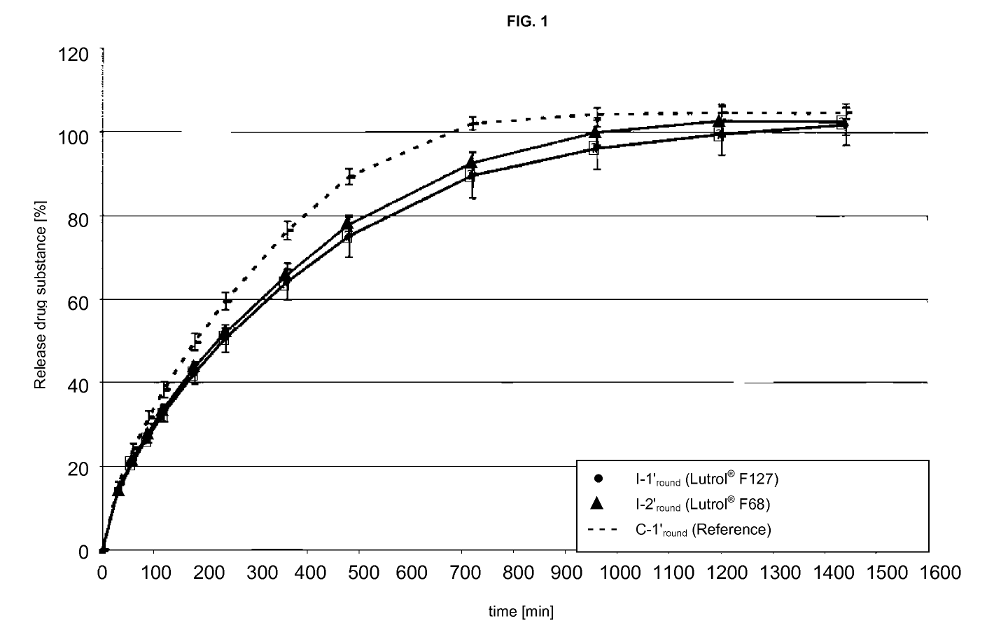

The in vitro release profiles of the pharmaceutical dosage forms according to Examples I-1′_{round}, I-2′_{round} and C-1′_{round} are displayed in FIG. 1.

### e) Tamper Resistance—Breaking Strength

All tablets did not break at a force of 1000 N, the upper measuring limit of the testing device.

### f) Tamper Resistance—Extractability

The extractable content of pharmacologically active compound was determined by
(v) subjecting a tablet (a) for 5 minutes in 5 mL of cold water, or (b) to boiling water and boiling the tablet for 5 minutes, respectively,
(vi) closing the vessel with aluminum foil, boiling extraction only,
(vii) drawing up the liquid into a syringe using a canula through a cigarette filter, and
(viii) determining the pharmacologically active compound content in the drawn liquid by HPLC analysis.

Each extraction was performed five-fold. The mean value of these extractions was assessed as the result.

The results are shown in the table here below:

| n = 5 [mean (min-max)] | C-1′_{round} | C-2′_{round} | I-1′_{round} | I-2′_{round} |
|---|---|---|---|---|
| extraction in cold water (20° C.) | 1.36% | 1.38% | 1.38% | 1.26% |
| | (1.2%-1.5%) | (1.3%-1.4%) | (1.3%-1.5%) | (1.1%-1.4%) |
| extraction in boiling water | 9.31% | 11.57% | 11.0% | 7.1% |
| | (8.4%-10.6%) | (10.9%-12.2%) | (10.1%-11.6%) | (5.2%-8.8%) |

It is clear from the above data that extraction in boiling water is substantially impeded by the nonionic surfactant contained in the pharmaceutical dosage form according to the invention. It appears that Poloxamer F68 (example I-2′_{round}) is more efficient in impeding hot liquid extraction than Poloxamer F127 (example I-1′_{round}).

### EXAMPLE 2

### a) Composition

Tablets having the following compositions were prepared:

| | C-3C-3 | | I-3 | |
|---|---|---|---|---|
| | Mg | wt.-% | mg | wt.-% |
| Tramadol HCl | 80.0 | 13.3 | 80.0 | 13.3 |
| Polyethylene Oxide M_{w} 7 × 10⁶ | 120.0 | 20.0 | 120.0 | 20.0 |
| HPMC 100,000 mPa · s | 90.0 | 15.0 | 90.0 | 15.0 |
| Macrogol 6,000 | 70.0 | 11.7 | 70.0 | 11.7 |
| Poloxamer 407 (Lutrol ® F127) | 120.0 | 20.0 | — | — |
| Poloxamer 188 (Lutrol ® F68) | — | — | 120.0 | 20.0 |
| Carbopol 71G | 120.0 | 20.0 | 120.0 | 20.0 |
| Σ | 600.0 | 100.0 | 600.0 | 100.0 |

### b) Hot-Melt Extrusion

The following extrusion parameters were adjusted and measured, respectively:

| | C-3C-3 | I-3 |
|---|---|---|
| diameter of die [mm] | 10 | 10 |
| throughput [kg/h] | 3.5 | 3.5 |
| melt temperature [° C.] | 115 | 116 |
| performance (%) | 36 | 30 |
| melt pressure [bar] | 6 | 5 |
| strand diameter [mm] | 8.4 | 10.3 |
| cutting length [mm] | 8.5-9.5 | 7-9 |

Crude extrudates having the following weights and dimensions were obtained:

| | | |
|---|---|---|
| n = 10 | C-3C-3 | I-3 |

| | | | |
|---|---|---|---|
| weight | min | 608 | 590 |
| [mg] | max | 627 | 634 |
| | average | 618 | 610 |
| length | min | 8.51 | 7.59 |
| [mm] | max | 8.81 | 8.31 |
| | average | 8.67 | 7.93 |
| diameter | min | 10.75 | 9.72 |
| [mm] | max | 11.11 | 10.86 |
| | average | 10.96 | 10.23 |

### c) Formation of Tablets from Extrudates

Tablets were manufactured from the crude extrudates by means of a round punch having the following dimensions (no engraving):

| Example | Form of punch |
|---|---|
| round | biconcave, round, diameter 12 mm, radius of curvature 9 mm |

Tablets having the following weights and dimensions were obtained:

| | | |
|---|---|---|
| | C-3C- | |
| n = 10 | 3_{round} | I-3_{round} |

| | | | |
|---|---|---|---|
| thickness | min | 6.55 | 6.71 |
| [mm] | max | 6.90 | 7.03 |
| | average | 6.79 | 6.88 |
| diameter | min | 11.65 | 11.67 |
| [mm] | max | 12.00 | 11.96 |
| | average | 11.81 | 11.83 |

### EXAMPLE 3

Example 1 was repeated under identical conditions and the properties of the inventive tablets (I-1′_{round}, I-2′_{round}, C-3 and I-3_{round}) were compared with comparators (C-1′_{round} and C-3_{round}).

### a) In-Vitro Release

| | C-1′_{round} | I-1′_{round} | I-2′_{round} | C-3C-3_{round} | I-3_{round} |
|---|---|---|---|---|---|
| measuring point | | | | | |
| Dissolution % | | | | | |
| (DS) | | | | | |
| after 60 min | 21 | 20 | 21 | 17 | 16 |
| after 120 min | 33 | 31 | 32 | 24 | 23 |
| after 480 min | 76 | 72 | 75 | 55 | 53 |
| after 720 min | 90 | 86 | 89 | 70 | 66 |
| after 1440 min | 101 | 97 | 98 | 95 | 89 |
| measuring point | | | | | |
| Dissolution % | | | | | |
| (0.1N HCl) | | | | | |
| after 60 min | 20 | 18 | 18 | 21 | 20 |
| after 120 min | 31 | 28 | 29 | 31 | 30 |
| after 480 min | 78 | 70 | 76 | 65 | 63 |
| after 720 min | 95 | 87 | 94 | 79 | 77 |
| after 1440 min | 102 | 99 | 106 | 98 | 96 |

### b) Tamper Resistance—Breaking Strength

| breaking | | | | C- | |
|---|---|---|---|---|---|
| strength [N] | C-1′_{round} | I-1′_{round} | I-2′_{round} | 3C-3_{round} | I-3_{round} |
| Sotax ®HT100 | ≧1000 N | ≧1000 N | ≧1000 N | 317 N | 551 N |

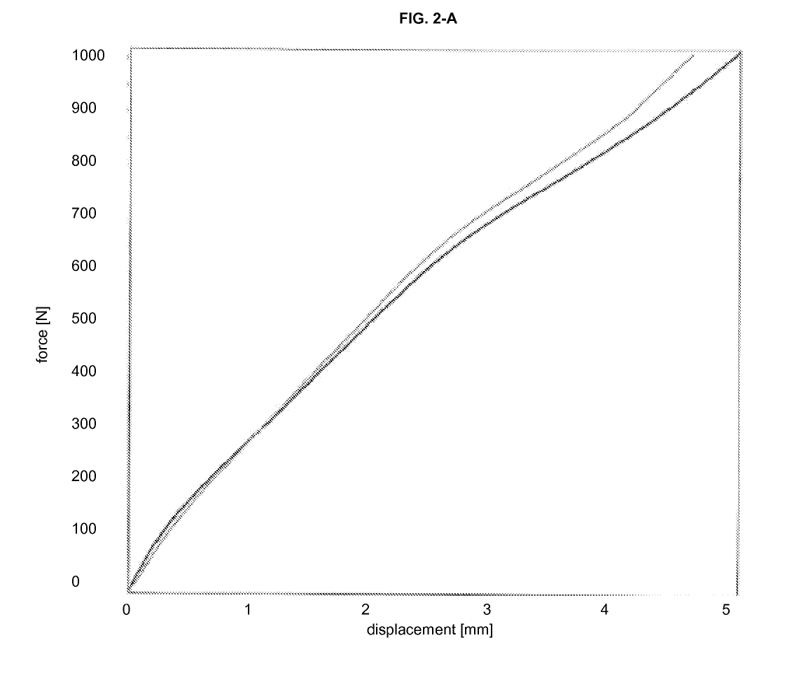

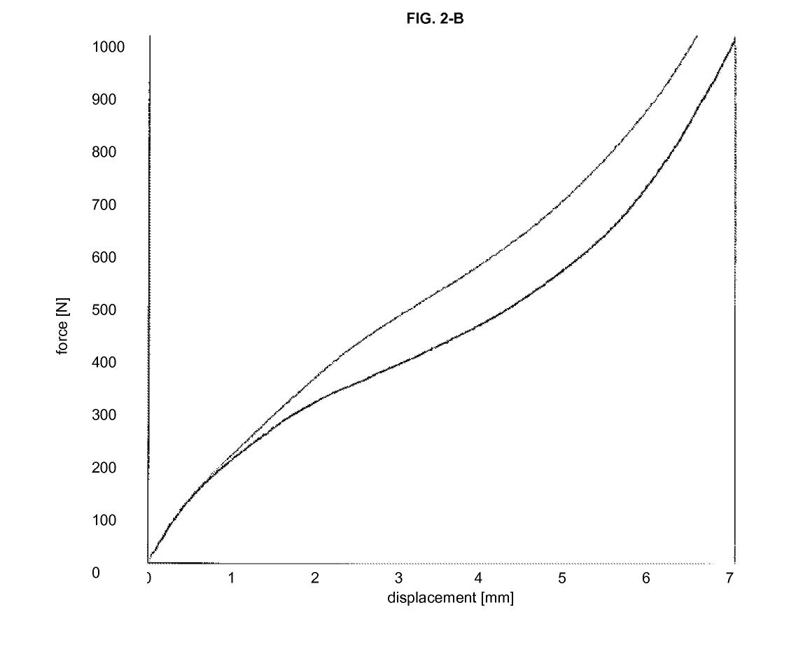

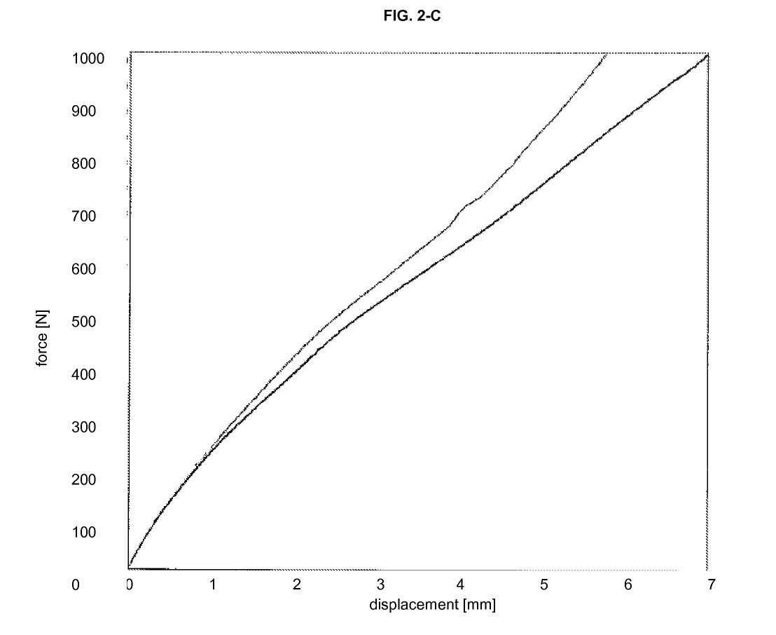

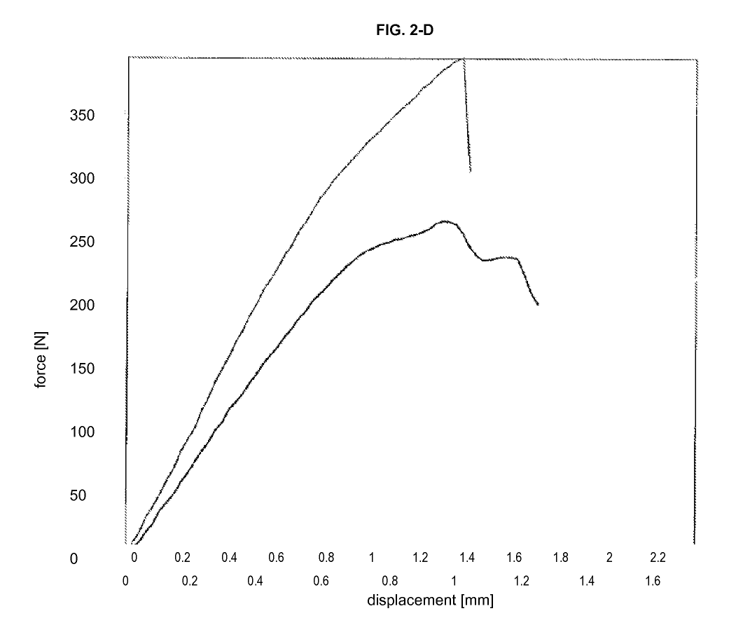

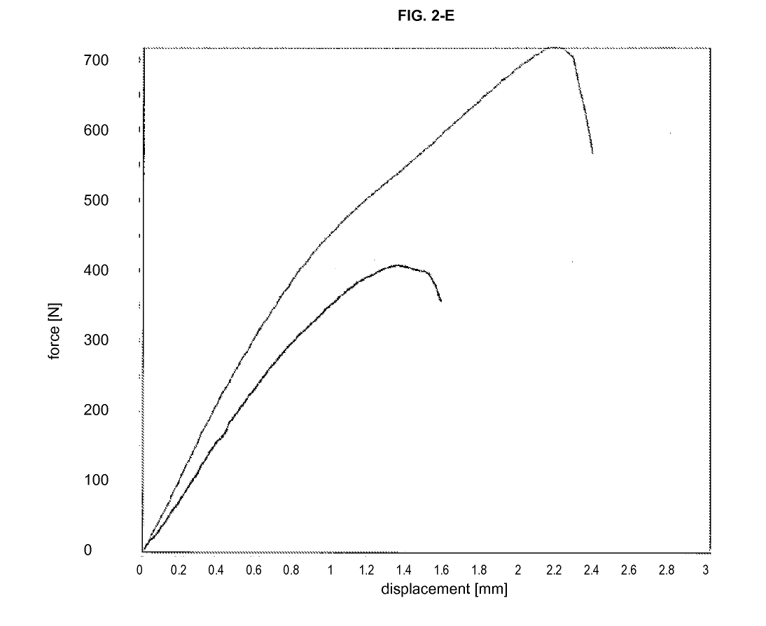

The force-displacement diagrams of examples C-1′_{round}, I-1′_{round}, I-2′_{round}, C-3 C-3_{round} and I-3_{round} are displayed as FIGS. 2-A, 2-B, 2-C, 2-D and 2-E, respectively.

### c) Tamper Resistance—Extractability

The extractable content of pharmacologically active compound was determined by
(iv) subjecting a tablet (a) for 30 minutes to 30 mL of solvent with continuous shaking, or (b) giving a tablet in 30 mL of purified water, heating the water until boiling and shaking for 30 minutes, during the slow cooling of the water,
(v) supplementing lost solvent, if any, and
(vi) determining the pharmacologically active compound content in the drawn liquid by HPLC analysis.

The results are shown in the table here below:

| content [wt.-%] | C-1′_{round} | I-1′_{round} | I-2′_{round} | C-3C-3_{round} | I-3_{round} |
|---|---|---|---|---|---|
| faultless tablet | 99.6 | 94.5 | 98.2 | 97.1 | 98.2 |
| extraction cold | 13.9 | 10.2 | 10.5 | 11.5 | 10.6 |
| water | | | | | |
| extraction boiling | 25.7 | 12.8 | 24.2 | 20.1 | 20.3 |
| water | | | | | |
| extraction water/ | 9.4 | 7.8 | 7.2 | 8.5 | 8.5 |
| ethanol 60/40 v/v | | | | | |

¹⁾household coffee mill, type Bosch MKM6000, 180W, type KM 13; grinding time: 2 minutes

### d) Tamper Resistance—Hammer Impact

The test was performed by means of a free falling weight testing device Type 40-550-001, 40-550-011 ff, Coesfeld GmbH & Co. KG, Germany. The following parameters were set:

Falling height: 1000 mm±1%
Falling weight: 500 g±2%
Form of falling weight: 25 mm×25 mm
Position of sample: loosely positioned in the center of the sample holder

The measuring result was qualified according to the following scale:
(A) tablet apparently undamaged
(B) tablet has been compressed but is widely undamaged
(C) tablet has been compressed and is lacerated at its edges
(D) tablet has been disrupted into several pieces
(E) tablet has been pulverized

The results are shown in the table here below:

| C-1′_{round} | I-1′_{round} | I-2′_{round} | C-3C-3_{round} | I-3_{round} |
|---|---|---|---|---|
| (B) | (C) | (A) | (D) | (D) |

### e) Tamper Resistance—Grindability

The tablets were treated by means of a commercially available household coffee mill, type Bosch MKM6000, 180W, type KM 13. Subsequently, the thus obtained material was analyzed by means of a sieving tower (Haver & Boecker, analysis sieve, diameter 50 mm) equipped with a bottom plate, displacement ring, lid, and 14 sieves the mesh sizes ranging from 0.045 mm to 4.000 mm, namely 0.045 mm; 0.063 mm; 0.090 mm; 0.125 mm; 0.180 mm; 0.250 mm; 0.355 mm; 0.500 mm; 0.710 mm; 1.000 mm; 1.400 mm; 2.000 mm; 2.800 mm; 4.000 mm. The amplitude was set to 1.5 mm. Sieving time was 10 min.

The test was performed in triplicate, the results (amount [%], average of n=3) after 2 minutes grinding are summarized in the table here below:

| 2 min | | | | | |
|---|---|---|---|---|---|
| grinding time | C-1′_{round} | I-1′_{round} | I-2′_{round} | C-3C-3_{round} | I-3_{round} |
| <0.045 | 0.00 | 0.00 | 0.00 | 0.00 | 0.00 |
| 0.045-0.063 | 0.00 | 0.55 | 0.54 | 0.51 | 0.00 |
| 0.063-0.090 | 0.00 | 0.00 | 0.00 | 0.65 | 0.00 |
| 0.090-0.125 | 0.00 | 1.23 | 0.54 | 1.80 | 1.10 |
| 0.125-0.180 | 0.00 | 0.00 | 0.00 | 2.95 | 2.32 |
| 0.180-0.250 | 0.00 | 0.55 | 0.57 | 5.26 | 5.73 |
| 0.250-0.355 | 1.42 | 2.08 | 2.20 | 10.02 | 13.68 |
| 0.355-0.500 | 1.42 | 5.61 | 2.16 | 11.82 | 13.57 |
| 0.500-0.710 | 3.72 | 8.00 | 6.46 | 13.77 | 16.27 |
| 0.710-1.000 | 6.56 | 12.44 | 8.15 | 10.16 | 14.67 |
| 1.000-1.400 | 14.54 | 19.41 | 16.83 | 9.65 | 13.96 |
| 1.400-2.000 | 27.42 | 19.11 | 28.74 | 7.19 | 10.15 |
| 2.000-2.800 | 16.60 | 8.11 | 28.98 | 5.51 | 5.13 |
| 2.800-4.000 | 16.97 | 18.60 | 4.83 | 3.72 | 2.42 |
| >4.000 | 11.35 | 4.32 | 0.00 | 16.99 | 1.00 |

It is clear from the above data that the dosage forms according to the invention have advantages compared to the reference with respect to extractability in cold water, ethanol and particularly hot water. In case of the inventive examples I-1′_{round} and I-2′_{round}, the remaining beneficial properties such as independence of release profile from pH value, high breaking strength and high impact resistance are not altered.

Including Carbopol into the inventive dosage form (C-3 I-3_{round}) leads to lower breaking strength compared to the reference example. However, the impact resistance and breaking strength, respectively, of inventive example C-3 I-3_{round} are still high enough to prevent crushing of the tablet by chewing and pulverization of the tablet by conventional means.

1. A pharmaceutical dosage form having a breaking strength of at least 500 N and comprising a pharmacologically active compound, a polyalkylene oxide having an average molecular weight of at least 200,000 g/mol, and a nonionic surfactant; wherein the content of the polyalkylene oxide is within the range of from 20 to 75 wt.-%, based on the total weight of the pharmaceutical dosage form.

2. The pharmaceutical dosage form according to claim 1, wherein the nonionic surfactant
(i) in pure water at a concentration of 25 wt.-% forms an aqueous dispersion having a viscosity η₁ at a temperature of 20° C. and a viscosity η₂ at a temperature of more than 20° C., where η₂>η₁; and/or
(ii) has an HLB value of at least 20, and/or
(iii) has a surface tension in 0.1% aqueous solution at 25° C. of at least 35 dynes/cm; and/or
(iv) has a viscosity of at most 4000 mPa·s, measured at 70° C. using a model LVF or LVT Brookfield viscosimeter.

3. The pharmaceutical dosage form according to claim 1, wherein the nonionic surfactant is a synthetic copolymer of ethylene oxide and propylene oxide.

4. The pharmaceutical dosage form according to claim 3, wherein the synthetic copolymer is
(i) a block copolymer according to general formula (I-a) 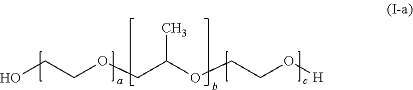
wherein a and c are each independently an integer of from 5 to 250, and b is an integer of from 10 to 100, or
(ii) a block copolymer according to general formula (I-b) 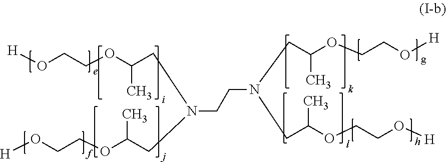
wherein e, f, g and h are each independently an integer of from 1 to 150, and i, j, k and l are each independently an integer of from 2 to 50

5. The pharmaceutical dosage form according to claim 1, wherein the content of the nonionic surfactant is within the range of from 0.1 to 30 wt.-%, relative to the total weight of the pharmaceutical dosage form.

6. The pharmaceutical dosage form according to claim 1, wherein the pharmacologically active compound is embedded in a prolonged release matrix comprising the polyalkylene oxide.

7. The pharmaceutical dosage form according to claim 1, which is configured for administration once daily or twice daily.

8. The pharmaceutical dosage form according to claim 1, wherein the content of the polyalkylene oxide is at least 30 wt.-%, based on the total weight of the pharmaceutical dosage form.

9. The pharmaceutical dosage form according to claim 1, wherein the polyalkylene oxide has an average molecular weight of at least 1,000,000 g/mol.

10. The pharmaceutical dosage form according to claim 1, wherein the pharmacologically active compound is an opioid selected from the group consisting of hydromorphone, oxycodone, oxymorphone, tramadol, tapentadol, and the physiologically acceptable salts thereof.

11. The pharmaceutical dosage form according to claim 1, which is thermoformed.

12. The pharmaceutical dosage form according to claim 11, which is hot-melt extruded.

13. The pharmaceutical dosage form according to claim 1, which is tamper-resistant.

14. The pharmaceutical dosage form according to claim 1, which contains a plasticizer.

15. The pharmaceutical dosage form according to claim 1, which contains an antioxidant.

16. A method of treating pain in a patient in need thereof, said method comprising administering to said patient a dosage form according to claim 10.
